(19) United States
(12) Patent Application Publication
(10) Pub. No.: US 2023/0052893 A1
(43) Pub. Date: Feb. 16, 2023

(54) A SYNERGISTIC BACTERICIDE AND BACTERIOSTATIC ORGANIC SANITIZING/DISINFECTANT/CLEANING FORMULATION

(71) Applicant: UNIVERSIDAD DE CONCEPCIÓN, Concepción (CL)

(72) Inventors: Luis AGUILAR CUENCA, Concepción (CL); Homero URRUTIA BRIONES, Concepción (CL); Nathaly RUIZ-TAGLE MOENA, Concepción (CL); Marjorie OBREQUE ESPINOZA, Concepción (CL)

(21) Appl. No.: 17/759,066

(22) PCT Filed: Jan. 22, 2021
(86) PCT No.: PCT/IB2021/050502

§ 371 Date: Jul. 19, 2022

Related U.S. Application Data
(60) Provisional application No. 62/964,538, filed on Jan. 22, 2020.

(51) Int. Cl. A01N 37/02; A61K 31/19

(52) U.S. Cl. CPC A01N 37/02; A61K 31/19

(57) ABSTRACT

A synergistic bactericide and bacteriostatic organic sanitizing/disinfectant/cleaning formulation of acetic acid and propionic acid or salts thereof, having a ratio acetate (A): propionate (P) from 4:100 to 20:1, dissolved in water and diluted as required, to a final concentration in the range of 0.5% w/v to 50% w/v of the mixture in the formulation. Particularly, the present formulation comprising 0.03-15% w/v of acetate buffer and 0.04-17.50% w/v and a q.s.p filtered drinking water, pH 5-6 to be used to the food industry as a Generally Recognized as Safe Compound (GRAS), and having a significantly increased sanitizing and sterilization effect on food as well inert hard, semi-hard and soft surfaces, being safe, environmentally friendly, of broad-spectrum and high efficiency. Such foods can be selected from harvested or fresh fruits and vegetables. Such hard surfaces can be selected from domestic surfaces including floors and furniture, industrial surfaces; and hospital surfaces including medical or dental tools and equipment surfaces. It is useful to combating and/or eliminating microorganisms selected from Listeria monocytogenes, Salmonella enterica, Escherichia coli, Staphylococcus aureus, Botrytis cinerea, Pseudomonas aeruginosa, Pseudomonas syringae, Klebsiella pneumoniae, Bacillus cereus, Bacillus subtilis as well other pathogenic microorganisms or its biofilms. It can be also used as antimicrobial additive.

### FIELD

The present invention relates to a synergistic bactericide and bacteriostatic organic sanitizing/disinfectant/cleaning formulation comprising a mixture of acetate (A) buffer and propionate (P) buffer at a ratio 4:100 to 20:1, preferably, such ratio A/P is selected from 4.3:100, 8.6:100, 17.2:100, 43:100, 86:100, 1.72:1, 4.30:1, 6.86:1, 8.6:1, 17.2:1 and 34.30:1 to a final concentration in the range from 0.5% w/v to 50% w/v of the mixture in the formulation, preferably, a final concentration selected from 0.67%, 3%, 3.38%, 5%, 11.25%, 15%, 17.14%, 20%, 25%, 34.29%, 35% or 50% w/v. Particularly, the present formulation comprising 0.03-15% w/v of acetate buffer and 0.04-17.50% w/v of propionate buffer and a q.s.p filtered drinking water, wherein q.s.p means quantity sufficient per. Preferably, 0.037-2.95% w/v of acetate buffer and 0.043-0.86% w/v of propionate buffer. More preferably, 0.37-5.17% w/v of acetate buffer and 0.043-6.06% w/v of propionate buffer. Even more preferably, 0.037-14.8 w/v of acetate buffer and 0.043-17.3% w/v of propionate buffer, 0.074-2.95% w/v of acetate buffer and 0.043-0.86% w/v of propionate buffer or 2.95% w/v of acetate buffer and 0.043-0.43% w/v of propionate buffer, and preferably at pH 5-6 to be used to the food industry as a Generally Recognized as Safe Compound (GRAS). Also, it has a significantly increased sanitizing and sterilization effect on food as well inert hard and semi-hard surfaces, being safe (innocuous), environmentally friendly, of broad-spectrum and highly efficiency. Such foods are selected from harvested or fresh fruits and vegetables, preferably blueberry and plum; and meat, preferably beef, chicken meat and salmon meat. Such hard surfaces can be selected from domestic surfaces including floors and furniture, industrial surfaces; and hospital surfaces including medical or dental tools and equipment surfaces. Such semi-hard surfaces can be selected from adhesive or non-adhesive sterile dressing, adhesive or non-adhesive absorbent sanitary napkin, and adhesive patch, which includes the present formulation. Soft surfaces are selected from health or wounded skin of human beings or animals and mucous, or plants and parts thereof or micropropagation material. The present synergistic organic sanitizing/disinfectant/cleaning formulation is specially, useful to combating and/or eliminating microorganisms selected from Listeria monocytogenes, Salmonella enterica, Escherichia coli, Staphylococcus aureus, Pseudomonas syringae, Botrytis cinerea, Pseudomonas aeruginosa as well other pathogenic microorganisms or its biofilms, having a significant role in combating and killing/eliminating them. Also, the present invention comprises a sanitizing/disinfectant/cleaning composition comprising the above mentioned formulation and excipients selected from natural or synthetic fragrances, natural or synthetic colorant agents, natural or synthetic surfactant, natural or synthetic organic additional active additives, natural or synthetic emulsifiers, natural or synthetic thickeners, among other pharmaceutically or food acceptable excipients. The present formulation or composition comprising the present formulation can be applied by direct contact, cold immersion or glazed, spray, fogging, aspersion, or immersion. The formulation can be also freeze-dried to be reconstituted before use.

### BACKGROUND TECHNIQUE

All the human being activities have in common a continuous and strong concern for combating and/or eliminating pathogen microorganisms from foods, meat, pets, human bodies, domestic surfaces, general surfaces of public places including stair railings, general surfaces of public or private vehicles, medical/dental devices, clothes, shoes, or the like. There are a huge number of sanitizing/disinfectant/cleaning formulations focused to comply such purpose/objective since pathogen microorganisms cause infections adversely affecting human health. Also, there is a number of medicaments but pathogen microorganism has learned to survive, in some way, spite of these solutions resulting from the human being efforts.

Particularly, a good cleaning of foods is so desirable and a fundamental target to be achieved during its processing and commercialization under different sale formats to guarantee a safe consumption. Thus, cleaning surfaces, equipment and tools in food processing installations is necessary due to all the industrial surfaces are susceptible to a contamination by pathogen agents which could be transferred to foods causing then serious health problems after ingestion.

Food-Borne Diseases (FBD) are caused from the ingestion of contaminated foods, contaminated by microorganisms or chemical substances affecting the health of the person or population group who/whom ate the same. Each year there is about of 3 million of dead persons around the world, either in developed or developing countries, by consumption of contaminated foods and water. Not a lesser number of persons can get sick by the same cause (FAO 2010). About 250 agents have been described as causing FBD including bacteria, virus, fungus, parasites, toxins and several types of chemical contaminant agents and heavy metal compounds. Some examples are Salmonella spp., Clostridium perfringens, Cryptosporidium sp., Hepatitis A virus, Aspergillus niger Linscott, A. J. (2011) Food-borne illnesses. Clinical Microbiology Newsletter. 33:41-45).

Between 2011 and 2014 years, the main identified and detected etiological agent to FBD in Chile, was Salmonella sp., achieving an average of 41%, having to 2014 a detected peak of 86% (MINSAL 2017a and 2017b. Ministry of Health (MINSAL) (2017a). Report of Outbreak Situation of Food Transmitted Diseases, epidemiological weeks 1 a 40//Ministry of Health (MINSAL) (2017b). Listerosis Quaterly Epidemiological Bulletin, weeks 1 to 39). The bacteria can produce diarrhea, fever and abdominal pains.

Similarly, bacteria Listeria monocytogenes has a big relevance as etiological agent to listeriosis, being able of causing fever, nausea or diarrhea in healthy people and having a dead rate of 20-30% in the most serious cases, in pregnant women, immunocompromised individuals and older people (Swaminathan, B., & Gerner-Smidt, P. (2007). The epidemiology of human listeriosis. Microbes and Infection. 9(10): 1236-1243).

Also it is known that microorganisms can form biofilms which can be formed over any surface of the industrial installation in processing plants, causing contamination of foods and water, corrosion and obstruction in equipment (Simoes, M., Simoes, L C., & Vieira, M J. (2010). A review of current and emergent biofilm control strategies. LWT-Food Science and Technology. 43(4): 573-583). Particularly, in the food industry, biofilms offering persistence of pathogen bacteria and increasing the risk of crossed contamination in foods, which involves a serious risk to consumers and subsequent economical lost due to contaminated food.

Based on this, new productive trends are focused to only satisfy the demand of food in the population rather than to implement more efficient sanitary practices. Thus, a food standard code has been established where the quality of a food is satisfactory—in relation to pathogen bacteria, when 25 g of food has no presence of Salmonella sp., Campylobacter sp., L. monocytogenes y E. coli H7:0157 (Gilbert, R J., Donovan, T., Little, C., Nye, K., Ribeiro, C D., Richards, J. & Bolton, F J. (2000). Guidelines for the microbiological quality of some ready to eat foods sampled at the point of sale. Communicable Disease and Public Health. 3(3):163-167.

All the surfaces vulnerable to be contaminated with pathogen microorganisms in food processing plants should be sanitized, which includes cleaning and disinfecting. A disinfecting process has as target destructing, inactivating or eliminating microorganisms causing human diseases (Wirtanen, G., & Salo, S. (2003). Disinfection in food processing—efficacy testing of disinfectants. Reviews in Environmental Science and Biotechnology. 2(2-4): 293-306).

Some disinfectants to be mentioned are phenolic compounds, chlorine, quaternary ammonium compounds, hypochlorous acid, peracetic acid, among others. Although disinfectant should be able to destroy a broad spectrum of pathogens, generally, the same frequently are more active against determined microorganisms, and specially under free living ways thus its activity can significantly vary as function of factors such as type of microorganism, administration/application form, dilution, temperature, pH, time of extension to the treatment and presence of other compounds as organic material (Meyer, B. (2003). Approaches to prevention, removal and killing of biofilms. International Biodeterioration & Biodegradation. 51(4): 249-253). Additionally, disadvantage such as toxicity, corrosion, stability, among others should be also taken into account. McDonnell, G., & Russell, A D. (1999). Antiseptics and disinfectants: activity, action, and resistance. Clinical Microbiology Reviews. 12: 147-179).

Biofilms can be defined as a sessile microorganism community adhered to a surface, embedded into a matrix of extracellular polymer substances which such microorganisms have produced and promotes an interaction between themselves. This is a surviving strategy since this structure protects to cells against to toxic compounds, microbicide agents, thermal stress and depredation. Further, after formed, cells increase a genetic exchange and show resistance to ultraviolet radiation and are highly strong against to antimicrobial treatments, which supports the importance of testing the efficacy of disinfectant substances against biofilms, in particular over stainless steel surfaces due to the resistance of this kind of surfaces to corrosion and durability since this is a preferred material to be used as contacting surfaces with foods.

Currently the food industry has experimented changes as result of a higher food demand, which in turn as resulting in an increase of volume production, mechanized operations, more processed foods and an increase of time and distance between production points, and higher consumption. Thus, the necessity of ensuring the preservation of foods to facilitate its distribution has increased (Berkowitz D E, Malagié M, Jensen G, Smith JGDL, Svagr J J, Spiegel J, et al., (2001) Industria alimentaria. Enciclopedia de salud y seguridad en el trabajo. 3^{a} ed. España: Ministerio de Trabajo y Asuntos Sociales. 67-2).

Disinfectants are very effectives in treating planktonic cells but the same are not biofilms due to microorganisms in biofilm can be 10 to 1.000 times more tolerant to chemical compounds tan planktonic cells (Mah, T F C., & O'toole, G A. (2001). Mechanisms of biofilm resistance to antimicrobial agents. Trends in microbiology. 9(1): 34-39), further currently there is a concern about of the resistance than microorganisms can acquire certain resistance to these products allowing the survival of pathogen bacteria to disinfection process and contaminating foods. In the other side, there is a negative perception respect to the use of these products in consumers due to the possibility of some toxic residues can be transferred to the foods and generating some inconvenience in the consumer (Giaouris, E., Heir, E., Hébraud, M., Chorianopoulos, N., Langsrud, S., Moøetrø, T., & Nychas, G. J. (2014). Attachment and biofilm formation by foodborne bacteria in meat processing environments: Causes, implications, role of bacterial interactions and control by alternative novel methods. Meat science. 97(3):298-309).

In relation to patent documents, US No. 2010/0239561 (Universidad Austral de Chile) discloses a formulation to control Listeria monocytogenes in the food industry, comprising 3 inactivated fermented lactic acid bacteria strains and nisin, acting as an antagonist action agent and bactericide against such pathogen causing listeriosis in human beings, disease transmitted through foods. This formulation is a GRAS agent by a nutrient competition between the lactic acid bacteria and the pathogen and reducing the pH over the surface where the formulation is applied.

U.S. Pat. No. 7,915,207 (Ecolab Inc) discloses an antimicrobial composition to be used for sanitizing foods, comprising GRAS compounds and food additives, particularly, the same comprises octanoic acid, an acidulant selected from citric acid and phosphoric acid, a buffer selected from citrate and phosphate, a sorbitan ester and a polyglucoside alkyl. This formulation can be applied to meat products to be ready to consumption.

US 2018/0216045 (ProNatural Brands LLC) discloses a cleaning and disinfecting composition containing an organic acid, a surfactant, and an alcohol, having a pH lower than 6. The organic acid can be selected from acetic acid, citric acid, ascorbic acid, fumaric acid, propionic acid, oxalic acid, malic acid, benzoic acid and carbonic acid. It is useful for disinfecting surfaces having in contact with pathogens E. coli, S. aureus, P. aeruginosa, K. pneumoniae, A. baumannii, Streptococcus groups A and D, Listeria and Salmonella.

WO 2017/180598 (WTI INC) discloses an antimicrobial composition to decontaminate surfaces in contact with foods, the composition is used against several pathogens including L. monocytogenes. The composition comprising an organic acid and a citric extract wherein the organic acid is selected from a group also including acetic acid and propanoic acid.

WO2014152734 (Hercules Inc) discloses a method for controlling a no-desired microorganism concentration in an aqueous system used in a fermentation process, comprising: (a) introducing a fermentable hydrocarbon in an aqueous solution; (b) introducing at least a yeast into such dissolution; (c) introducing a first organic acid and a second organic acid in the aqueous system in which the first organic acid is citric acid or a salt thereof, the second organic acid is propionic acid or a salt thereof and the ratio of first organic acid or a salt thereof to the second organic acid or salt thereof is from 32:1 up to 1:32, wherein the first organic acid having a concentration of at least 100 ppm in the aqueous system.

Based on what is previously mentioned, there is still a necessity of counting with new strategies to combating pathogen in the food area, including more eco-friendly alternatives and also be able to control biofilms.

Also, it is needed a composition which can offer a broad spectrum to disinfecting and versatile to a kind of material over which it can be applied and the form in which the same can be administered/applied/supplied to provide its antimicrobial disinfecting action, for example, as a composition forming an adherent polymeric film or can be sprayed on a substrate surface or added into a primary composition to provide a sustained antimicrobial disinfecting action upon contact with microorganisms for prolonged periods, with or without the necessity for reapplication.

The present formulation can be also used as antimicrobial additive as well as an environment sanitizer to be sprayed.

### BRIEF DESCRIPTION OF DRAWINGS

FIG. 1: Counting of biofilm cells to L. monocytogenes exposed a disinfectant and peracetic acid by 30 minutes.

FIG. 2: Counting of biofilm cells to E. coli exposed a disinfectant and peracetic acid by 30 minutes.

FIG. 3: Counting of biofilm cells to S. enterica exposed a disinfectant and peracetic acid by 30 minutes.

FIG. 4: Counting of biofilm cells to S. aureus exposed a disinfectant and peracetic acid by 30 minutes.

FIG. 5: Counting of bacteria (CFU/ml) recovered from 24-hour E. coli ATCC 25932 biofilms exposed for 30 min to synergistic mixtures of acetate and propionate solutions in proportions ranging from 17.2:1 to 0.043:1, at 5%, 15% and 50% concentrations.

FIG. 6: Counting of bacteria (CFU/ml) recovered from 24-hour Salmonella enterica ATCC 13076 biofilms exposed for 30 min to synergistic mixtures of acetate and propionate solutions in proportions ranging from 17.2:1 to 0.043:1, at 5%, 15% and 50% concentrations.

FIG. 7: Counting of bacteria (CFU/ml) recovered from 24-hour Staphylococcus aureus ATCC 6538P biofilms exposed for 30 min to synergistic mixtures of acetate and propionate solutions in proportions ranging from 17.2:1 to 0.043:1, at 5%, 15% and 50% concentrations.

FIG. 8: Counting of bacteria (CFU/ml) recovered from 24-hour Listeria monocytogenes ATCC 19115 biofilms exposed for 30 min to synergistic mixtures of acetate and propionate solutions in proportions ranging from 17.2:1 to 0.043:1, at 5%, 15% and 50% concentrations.

FIG. 9: Incidence in blueberry fruits at 4° C., fruits showing signs of pathogens (Botrytis cinerea) were counted. The treatments correspond to the formulation in a 17.2:1 portion at 3.38% and 25%.

FIG. 10: Incidence in blueberry fruits at 4° C., fruits showing signs of pathogens (Botrytis cinerea) were counted. The treatments correspond to the formulation in a 6.8:1 portion at 3.38%, 11.25% and 25%.

FIG. 11: Incidence in blueberry fruits at 4° C., fruits showing signs of pathogens (Botrytis cinerea) were counted. The treatments correspond to the formulation in a 1.72:1 portion at 3.38% and 25%.

FIG. 12: Incidence in blueberry fruits at 4° C., fruits showing signs of pathogens (Botrytis cinerea) were counted. The treatments correspond to the formulation in a 0.043:1 portion at 3.38% and 25%.

FIG. 13: Incidence in blueberry fruits at 4° C., fruits showing signs of pathogens (Penicillium spp.) were counted. The treatments correspond to the formulation in a 17.2:1 portion at 3.38% and 25%.

FIG. 14: Incidence in blueberry fruits at 4° C., fruits showing signs of pathogens (Penicillium spp.) were counted. The treatments correspond to the formulation in a 6.8:1 portion at 3.38% and 25%.

FIG. 15: Incidence in blueberry fruits at 4° C., fruits showing signs of pathogens (Penicillium spp.) were counted. The treatments correspond to the formulation in a 1.72:1 portion at 3.38% and 25%.

FIG. 16: Incidence in blueberry fruits at 4° C., fruits showing signs of pathogens (Penicillium spp.) were counted. The treatments correspond to the formulation in a 0.043:1 portion at 3.38% and 25%.

FIG. 17: Incidence in plum fruits at 4° C., fruits showing signs of pathogens (Botrytis cinerea) were counted. The treatments correspond to the formulation in a 17.2:1 portion at 3.38% and 25%.

FIG. 18: Incidence in plum fruits at 4° C., fruits showing signs of pathogens (Botrytis cinerea) were counted. The treatments correspond to the formulation in a 6.8:1 portion at 3.38%, 11.25% and 25%.

FIG. 19: Incidence in plum fruits at 4° C., fruits showing signs of pathogens (Botrytis cinerea) were counted. The treatments correspond to the formulation in a 1.72:1 portion at 3.38% and 25%.

FIG. 20: Incidence in plum fruits at 4° C., fruits showing signs of pathogens (Botrytis cinerea) were counted. The treatments correspond to the formulation in a 0.043:1 portion at 3.38% and 25%.

FIG. 21: Incidence in plum fruits at 4° C., fruits showing signs of pathogens (Penicillium spp.) were counted. The treatments correspond to the formulation in a 17.2:1 portion at 3.38% and 25%.

FIG. 22: Incidence in plum fruits at 4° C., fruits showing signs of pathogens (Penicillium spp.) were counted. The treatments correspond to the formulation in a 6.8:1 portion at 3.38% and 25%.

FIG. 23: Incidence in plum fruits at 4° C., fruits showing signs of pathogens (Penicillium spp.) were counted. The treatments correspond to the formulation in a 1.72:1 portion at 3.38% and 25%.

FIG. 24: Incidence in plum fruits at 4° C., fruits showing signs of pathogens (Penicillium spp.) were counted. The treatments correspond to the formulation in a 0.043:1 portion at 3.38% and 25%.

FIG. 25: Incidence in tangerine fruits at 4° C., fruits showing signs of pathogens (Botrytis cinerea) were counted. The treatments correspond to the formulation in a 17.2:1 portion at 3.38% and 25%.

FIG. 26: Incidence in tangerine fruits at 4° C., fruits showing signs of pathogens (Botrytis cinerea) were counted. The treatments correspond to the formulation in a 6.8:1 portion at 3.38%, 11.25% and 25%.

FIG. 27: Incidence in tangerine fruits at 4° C., fruits showing signs of pathogens (Botrytis cinerea) were counted. The treatments correspond to the formulation in a 1.72:1 portion at 3.38% and 25%.

FIG. 28: Incidence in tangarine fruits at 4° C., fruits showing signs of pathogens (Botrytis cinerea) were counted. The treatments correspond to the formulation in a 0.043:1 portion at 3.38% and 25%.

FIG. 29: Incidence in tangarine fruits at 4° C., fruits showing signs of pathogens (Penicillium spp.) were counted. The treatments correspond to the formulation in a 17.2:1 portion at 3.38% and 25%.

FIG. 30: Incidence in tangarine fruits at 4° C., fruits showing signs of pathogens (Penicillium spp.) were counted. The treatments correspond to the formulation in a 6.8:1 portion at 3.38% and 25%.

FIG. 31: Incidence in tangarine fruits at 4° C., fruits showing signs of pathogens (Penicillium spp.) were counted. The treatments correspond to the formulation in a 1.72:1 portion at 3.38% and 25%.

FIG. 32: Incidence in tangarine fruits at 4° C., fruits showing signs of pathogens (Penicillium spp.) were counted. The treatments correspond to the formulation in a 0.043:1 portion at 3.38% and 25%.

FIG. 33: Effect of the disinfectant treatment over bacteria counting to 0 and 3 days in chicken meat at 6.8:1 ratio. A) Counting in general TSA medium and B) Counting of enterobacteria, Negative Control: initial counting (n=3).

FIG. 34: Effect of the disinfectant treatment over bacteria counting to 0, 3, 8 and 12 days in chicken at 0.043:1 ratio. A) Counting in general TSA medium and B) Counting of enterobacteria, Negative Control: initial counting (n=3).

FIG. 35: Effect of the disinfectant treatment over bacteria counting to 0, 3, 8 and 12 days in chicken at 0.172:1 ratio. A) Counting in general TSA medium and B) Counting of enterobacteria, Negative Control: initial counting (n=3).

FIG. 36: Effect of the disinfectant treatment over bacteria counting to 0, 3, 8 and 12 days in chicken at 1.72:1 ratio. A) Counting in general TSA medium and B) Counting of enterobacteria, Negative Control: initial counting (n=3).

FIG. 37: Effect of the disinfectant treatment over bacteria counting to 0, 3, 8 and 12 days in chicken at 17.2:1 ratio. A) Counting in general TSA medium and B) Counting of enterobacteria, Negative Control: initial counting (n=3).

FIG. 38: Effect of the disinfectant treatments over the bacteria counting to 0 and 3 days in beef at 6.8:1 ratio. A) Counting in genera TSA medium and B) Counting of enterobacteria, Negative Control: initial counting (n=3).

FIG. 39: Effect of the disinfectant treatment over bacteria counting to 0, 3, 8 and 12 days in beef at 0.043:1 ratio. A) Counting in general TSA medium and B) Counting of enterobacteria, Negative Control: initial counting (n=3).

FIG. 40: Effect of the disinfectant treatment over bacteria counting to 0, 3, 8 and 12 days in beef at 0.172:1 ratio. A) Counting in general TSA medium and B) Counting of enterobacteria, Negative Control: initial counting (n=3).

FIG. 41: Effect of the disinfectant treatment over bacteria counting to 0, 3, 8 and 12 days in beef at 1.7:1 ratio. A) Counting in general TSA medium and B) Counting of enterobacteria, Negative Control: initial counting (n=3).

FIG. 42: Effect of the disinfectant treatment over bacteria counting to 0, 3, 8 and 12 days in beef at 17.2:1 ratio. A) Counting in general TSA medium and B) Counting of enterobacteria, Negative Control: initial counting (n=3).

FIG. 43: Effect of the disinfectant treatment over bacteria counting to 0 and 3 days in salmon fish at 6.8:1. A) Counting in general TSA medium and B) Counting of enterobacteria, Negative Control: Initial Counting (n=3).

FIG. 44: Effect of the disinfectant treatment over bacteria counting to 0, 3, 8 and 12 days in salmon fish at 0.043:1. A) Counting in general TSA medium and B) Counting of enterobacteria, Negative Control: Initial Counting (n=3).

FIG. 45: Effect of the disinfectant treatment over bacteria counting to 0, 3, 8 and 12 days in salmon fish at 0.172:1. A) Counting in general TSA medium and B) Counting of enterobacteria, Negative Control: Initial Counting (n=3).

FIG. 46: Effect of the disinfectant treatment over bacteria counting to 0, 3, 8 and 12 days in salmon fish at 1.72:1. A) Counting in general TSA medium and B) Counting of enterobacteria, Negative Control: Initial Counting (n=3).

FIG. 47: Effect of the disinfectant treatment over bacteria counting to 0, 3, 8 and 12 days in salmon fish at 17.2:1. A) Counting in general TSA medium and B) Counting of enterobacteria, Negative Control: Initial Counting (n=3).

FIG. 48: Effect of the disinfectant treatments over the bacteria counting to 0 and 3 days in E. coli inoculated salmon meat. A) Counting in general TSA medium and B) Counting of E. coli, Negative Control: Initial Counting (n=3).

FIG. 49: Effect of disinfectant treatments over the bacteria counting to 0 and 3 days in E. coli inoculated beef. A) Counting in general TSA medium and B) Counting enterobacteria, Negative Control: Initial Counting (n=3).

FIG. 50: Effect of the disinfectant treatments over the bacteria counting to 0 and 3 days in E. coli inoculated chicken meat. A) Counting general TSA medium and B) Counting of E. coli, Negative Control: Initial Counting (n=3).

FIG. 51: FIG. 51: A) E. coli and B) S. aureus (CFU/support) recovered from different supports: aquaculture nets; glass; aluminum can; gauze pads; polypropylene and stainless steel coupons after treatment for 30 minutes with synergistic mixtures of acetate and propionate solutions in the proportions 17.2:1, 1.72:1, 0.172:1 and 0.043:1. Each treatment was exposed to 15 and 35% concentrations; only propylene was exposed to 50%.

### DESCRIPTION OF THE INVENTION

The present synergistic organic sanitizing/disinfectant/cleaning formulation comprising a mixture of organic acid and salts thereof, all compounds type GRAS (Generally Recognized as Safe), being such organic compounds acetic acid and propionic acid.

The present synergistic organic formulation is preferably oriented to the food industry due to the same comprises compounds type GRAS, can be in direct contact with food, being innocuous to the environmental and consumer, further its use does not generate toxic residues.

The present synergistic organic formulation comprising a mixture of acetate (A) buffer and propionate (P) buffer at a ratio 4:100 to 20:1, preferably, such ratio A/P is selected from 4.3:100, 8.6:100, 17.2:100, 43:100, 86:100, 1.72:1, 4.30:1, 6.86:1, 8.6:1, 17.2:1 and 34.30:1 to a final concentration in the range from 0.5% w/v to 50% w/v of the mixture in the formulation, preferably, a final concentration selected from 0.67%, 3%, 3.38%, 5%, 11.25%, 15%, 17.14%, 20%, 25%, 34.29%, 35% or 50% w/v. Particularly, the present formulation comprising 0.03-15% w/v of acetate buffer and 0.04-17.50% w/v of propionate buffer and a q.s.p filtered drinking water. Preferably, 0.037-2.95% w/v of acetate buffer and 0.043-0.86% w/v of propionate buffer. More preferably, 0.37-5.17% w/v of acetate buffer and 0.043-6.06% w/v of propionate buffer. Even more preferably, 0.037-14.8 w/v of acetate buffer and 0.043-17.3% w/v of propionate buffer, 0.074-2.95% w/v of acetate buffer and 0.043-0.86% w/v of propionate buffer or 2.95% w/v of acetate buffer and 0.043-0.43% w/v of propionate buffer.

The present synergistic organic formulation can be prepared at a pH higher to commercial acid disinfectants (pH lower 4), thus the same is lower corrosive those known in the prior art, further having a pH range within different processed foods as sausages, among others. Preferably, the present proportions of this synergistic organic formulation having a pH values that fluctuates from 1 to 6, preferably pH values ranging 3-6, more preferably a pH value of 3, to be used to the food industry as a Generally Recognized as Safe Compound (GRAS)

The present synergistic organic disinfectant formulation can be used in different dilutions to satisfy particular necessities of the industry. At low concentrations the present formulation can be directly used over foods during its processing, packaging or to the prior cleaning to the food preparation to prevent the infection with pathogens and concentrations higher is able to remove bacteria biofilms which are a problem in different surfaces of the food industry.

At higher concentrations the present formulation can be directly used by immersion or by spraying over surfaces to disinfect.

The use of the present synergistic organic formulation allows controlling or eliminating the presence of pathogen microorganisms avoiding the presence of the same in foods or surfaces of a processing plant, in this way outbreaks or cases of sick persons for food contaminated with pathogen bacteria are avoided, economical lost to producers by withdrawal of contaminated products from the market, damaging the imagen of the involved company and losing the confidence of the consumer. Further, the present formulation can be used to clean foods and vegetables in houses or commercial places of preparing foods.

The present synergistic disinfectant formulation is able to act as bactericide or bacteriostatic against to different gram negative and gram-positive pathogens, some examples of them are Listeria monocytogenes, Salmonella enterica, Escherichia coli, Escherichia coli O157:H7, Staphylococcus aureus, Botrytis cinerea, Penicillium spp., Pseudomonas syringae, Bacillus cereus, Klebsiella pneumoniae y Pseudomonas aeruginosa.

Other commercial formulations are used over surfaces of the food industry but then the same should be removed to avoid the food contamination since they are toxic and some residues can affect the consumer. The present synergistic formulation has a low toxicity according to toxicological classification (Category V) under concentrated formulation thus to low concentrations the risk of toxicity decreases.

The present synergistic bactericide and bacteriostatic organic disinfectant/sanitizing/cleaning formulation can be further prepared to comprise natural or synthetic fragrances, natural or synthetic colorant agents, natural or synthetic surfactant, natural or synthetic organic additional active additives, natural or synthetic emulsifiers, natural or synthetic thickeners, among other pharmaceutically or food acceptable excipients.

The present synergistic organic disinfectant/sanitizing/cleaning formulation has a bactericide and bacteriostatic effect and a significantly increased sanitizing and sterilization effect on food as well inert hard, semi-hard and soft surfaces, being safe (innocuous), environmentally friendly, of broad-spectrum and high efficiency. Such foods are selected from harvested or fresh fruits and vegetables and meat. Such hard surfaces can be selected from domestic surfaces including floors and furniture, industrial surfaces; and hospital surfaces including medical or dental tools and equipment surfaces. Such semi-hard surfaces can be selected from adhesive or non-adhesive sterile dressing, adhesive or non-adhesive absorbent sanitary napkin, and adhesive patch or adhesive or non-adhesive bandages, which includes the present formulation. Soft surfaces are selected from health or wounded skin of human beings or animals and mucous, or plants and parts thereof.

Also, it is needed a composition which can offer a broad spectrum to disinfecting and versatile to a kind of material over which it can be applied and the form in which the same can be administered/applied/supplied to provide its antimicrobial disinfecting action, for example, as a composition forming an adherent polymeric film or can be sprayed on a substrate surface or added into a primary composition to provide a sustained antimicrobial disinfecting action upon contact with microorganisms for prolonged periods, with or without the necessity for reapplication. The present formulation can be also used as antimicrobial additive an environment sanitizer to be sprayed. The present formulation or a composition comprising the present formulation can be applied by direct contact, cold immersion or glazed, spray, fogging, aspersion, or immersion. The formulation can be also freeze-dried to be reconstituted before use.

It is an objective of the present invention a synergistic bactericide and bacteriostatic organic sanitizing/disinfectant/cleaning formulation comprising a mixture of acetic acid and propionic acid or salts thereof at a ratio acetate:propionate is in the range of 4:100 to 20:1, preferably, 4.3:100, 8.6:100, 17.2:100, 43:100, 59:100, 86:100, 1.72:1, 4.30:1, 6.86:1, 8.6:1, 17.2:1 and 34.30 The synergistic bactericide and bacteriostatic organic sanitizing/disinfectant/cleaning formulation having a concentration of such mixture of acetic acid and propionic acid or salts thereof in a range of 0.5% w/v to 50% w/v of the mixture in the formulation, preferably, 0.67%, 3%, 3.38%, 5%, 11.25%, 15%, 17.14%, 20%, 25%, 34.29%, 35%, or 50% w/v. Preferably, the present formulation having a pH in the range from 3 to 6.

It is another objective of the present invention the use of the above mentioned formulation to sanitizing and sterilization food as well surfaces. Preferably, its use a Generally Recognized as Safe Compound (GRAS). More preferably, its use to sanitizing and sterilization of food selected from one or more of blueberry, tangerine and plum, and meat including beef, chicken meat and salmon meat. As well, its use to sanitizing and sterilization of surfaces selected from one or more of floors and furniture, industrial surfaces; and hospital surfaces including medical or dental tools and equipment surfaces. The present formulation further comprising natural or synthetic fragrances, natural or synthetic colorant agents, natural or synthetic surfactant, natural or synthetic organic additional active additives, natural or synthetic emulsifiers or natural or synthetic thickeners and can be freeze-dried by conventional technique to be reconstituted before use.

It is another objective of the present invention the use of the synergistic organic sanitizing/disinfectant/cleaning formulation to combating and/or eliminating microorganisms including Listeria monocytogenes, Salmonella enterica, Escherichia coli, Staphylococcus aureus, Botrytis cinerea, Pseudomonas aeruginosa, Klebsiella pneumoniae, Bacillus cereus o Pseudomonas syringae or its biofilms.

To determine the synergistic activity of an antimicrobial compound fractional inhibitory concentration (FIC) is used (Ec. 1):

FIC=[a]/CMIa+[b]/CMIb (Ec. 1);

wherein [a] is the concentration of a first active compound from the mixture/combination and [b] is the concentration of a second active compound of such mixture/combination when used together, each one divided by its minimal concentration (CMI value) when used alone. Thus, a synergism is confirmed to FIC<1.0, an additive effect is confirmed when FIC=1.0 and antagonism is confirmed to FIC>1 (EP1332675B2, Kull, F. C.; Eisman, P. C.; Sylwestrowicz, H. D. and Mayer, R. L., in Applied Microbiology 9:538-541 (1961))

### EXAMPLES

### Example 1: Preparation of the Formulation

To prepare the present synergistic formulations two different organic acids considered as Generally Recognized as Safe (GRAS) substances were selected. These two different organic acids are acetic acid and propionic acid and salts thereof.

All the presents synergistic formulations were prepared in a buffered solution at a range of pH 3-6, if otherwise stated, the formulation was tested at pH 5-6. which also is a pH acceptable to foods. Further, the characteristic odor of acetic acid or acetate buffer was masked by propionic acid or propionate buffer, being the resulting mixture more friendly environmentally.

Thus the present synergistic formulation was prepared mixing an acetate buffer and a propionate buffer at a rate 17.2:1, 6.8:1, 1.72:1, 0.172:1, 0.043:1, in filtered drinking water, and then heated at 60° C. and stirred up to achieve a homogenized mixture. This formulation shows a concentration which results from the sum of the concentration of both buffers (acetate and propionate). From this stock solution dilutions were prepared to perform antimicrobial tests against planktonic bacteria under liquid culture, and biofilms in surface of stainless steel and in direct contact with foods.

### Example 2: Use of the Formulation in Planktonic Cells

### 2.1 MIC-MBC

The efficacy of the formulation was evaluated through minimal inhibitory concentration (MIC) and minimal bactericide concentration (MBC) against pathogens E. coli ATCC 25932, L. monocytogenes ATCC 19115; Staphylococcus aureus ATCC 6538P, Salmonella enterica ATCC 13076. To determine MIC a final concentration of 10⁵ UFC/ml bacteria was inoculated at 96 multiwell plates containing 180 μl of different concentrations of disinfectant in TSB medium and the presence or absence of growing was observed to 18-20 hrs of incubation, then to MBC 100 μl was taken from wells and re-inoculated in 5 ml of liquid nutritive TSB medium, finally tubes without growth was registered after 20 hrs. The range of disinfectant concentrations was 0.037-5.17% w/v to acetate buffer and 0.043-6.06% w/v to propionate buffer. These concentrations are obtained from a stock solution which is diluted with filtered water.

From the results it is noted that between 0.74-3.69% w/v of acetate buffer and 0.43-3.46% w/v of propionate buffer was needed by separate to eliminate the above-mentioned pathogens. In combination it is required 0.037-2.95% acetate buffer and 0.043-0.86% propionate buffer to achieve the bactericide effect (Table 1-4). This activity allows to know the antimicrobial activity of the present disinfectant after a prolongated exposition period of time (18-20 hrs), from which a minimal dose was determined and promoting the performance of subsequent tests to determine efficacy.

TABLE 1
Results of MIC (+) y MBC (++) to L. monocytogenes in
a mixture of acetate buffer and propionate buffer by separate and
mixed, (−) no growth inhibition is noted, (+) growth inhibition is noted.

| Acetate | | | | | | | | | |
|---|---|---|---|---|---|---|---|---|---|
| w/v | | | | | | | | | |
| 5.17% | ++ | ++ | ++ | ++ | ++ | ++ | ++ | ++ | |
| 3.69% | ++ | ++ | ++ | ++ | ++ | ++ | ++ | ++ | |
| 2.95% | ++ | ++ | ++ | ++ | ++ | ++ | ++ | ++ | |
| 0.74% | ++ | ++ | ++ | ++ | ++ | ++ | ++ | ++ | |
| 0.37% | + | + | ++ | ++ | ++ | ++ | ++ | ++ | |
| 0.074% | + | + | ++ | ++ | ++ | ++ | ++ | ++ | |
| 0.037% | + | + | + | ++ | ++ | ++ | ++ | ++ | |
| 0% | − | + | + | ++ | ++ | ++ | ++ | ++ | |
| | 0 | 0.043% | 0.086% | 0.43% | 0.86% | 3.46% | 4.33% | 6.06% | Propionate |
| | | | | | | | | | w/v |

TABLE 2
Results of MIC (+) y MBC (++) of S. enterica in a mixture
of acetate buffer and propionate buffer by separate and mixed,
(−) no growth inhibition is noted, (+) growth inhibition is noted.

| Acetate | | | | | | | | | |
|---|---|---|---|---|---|---|---|---|---|
| w/v | | | | | | | | | |
| 5.17% | ++ | ++ | ++ | ++ | ++ | ++ | ++ | ++ | |
| 3.69% | ++ | ++ | ++ | ++ | ++ | ++ | ++ | ++ | |
| 2.95% | ++ | ++ | ++ | ++ | ++ | ++ | ++ | ++ | |
| 0.74% | + | + | + | ++ | ++ | ++ | ++ | ++ | |
| 0.37% | + | + | + | + | ++ | ++ | ++ | ++ | |
| 0.074% | + | + | + | + | + | ++ | ++ | ++ | |
| 0.037% | + | + | + | + | + | ++ | ++ | ++ | |
| 0% | − | + | + | + | + | ++ | ++ | ++ | |
| | 0 | 0.043% | 0.086% | 0.43% | 0.86% | 3.46% | 4.33% | 6.06% | Propionate |
| | | | | | | | | | w/v |

TABLE 3
Results of MIC (+) y MBC (++) of E. coli in a mixture
of acetate buffer and propionate buffer by separate and mixed,
(−) no growth inhibition is noted, (+) growth inhibition is noted.

| Acetate | | | | | | | | | |
|---|---|---|---|---|---|---|---|---|---|
| w/v | | | | | | | | | |
| 5.17% | ++ | ++ | ++ | ++ | ++ | ++ | ++ | ++ | |
| 3.69% | ++ | ++ | ++ | ++ | ++ | ++ | ++ | ++ | |
| 2.95% | + | + | + | ++ | ++ | ++ | ++ | ++ | |
| 0.74% | + | + | + | + | + | ++ | ++ | ++ | |
| 0.37% | + | + | + | + | + | ++ | ++ | ++ | |
| 0.074% | + | + | + | + | + | ++ | ++ | ++ | |
| 0.037% | + | + | + | + | + | ++ | ++ | ++ | |
| 0% | − | + | + | + | + | ++ | ++ | ++ | |
| | 0 | 0.043% | 0.086% | 0.43% | 0.86% | 3.46% | 4.33% | 6.06% | Propionate |
| | | | | | | | | | w/v |

TABLE 4
Results of MIC (+) y MBC (++) of S. aureus in a mixture
of acetate buffer and propionate buffer by separate and mixed,
(−) no growth inhibition is noted, (+) growth inhibition is noted.

| Acetate | | | | | | | | | |
|---|---|---|---|---|---|---|---|---|---|
| w/v | | | | | | | | | |
| 5.17% | ++ | ++ | ++ | ++ | ++ | ++ | ++ | ++ | |
| 3.69% | ++ | ++ | ++ | ++ | ++ | ++ | ++ | ++ | |
| 2.95% | ++ | ++ | ++ | ++ | ++ | ++ | ++ | ++ | |
| 0.74% | + | ++ | ++ | ++ | ++ | ++ | ++ | ++ | |
| 0.37% | + | + | + | ++ | ++ | ++ | ++ | ++ | |
| 0.074% | + | + | + | ++ | ++ | ++ | ++ | ++ | |
| 0.037% | + | + | + | + | ++ | ++ | ++ | ++ | |
| 0% | − | + | + | + | + | ++ | ++ | ++ | |
| | 0 | 0.043% | 0.086% | 0.43% | 0.86% | 3.46% | 4.33% | 6.06% | Propionate |
| | | | | | | | | | w/v |

TABLE 5
Ratios A/P

| Acetate | | | | | | | | |
|---|---|---|---|---|---|---|---|---|
| w/v | A/P | A/P | A/P | A/P | A/P | A/P | A/P | |
| 5.17% | 1.20 | 60.11 | 1.20 | 60.11 | 1.51 | 1.18 | 0.85 | |
| 3.69% | 8.58 | 42.91 | 8.58 | 42.91 | 1.08 | 0.85 | 0.61 | |
| 2.95% | 6.86 | 34.30 | 6.86 | 3.43 | 0.86 | 0.68 | 0.49 | |
| 0.74% | 17.2 | 8.60 | 1.72 | 0.86 | 0.22 | 0.172 | 0.12 | |
| 0.37% | 8.60 | 4.30 | 0.86 | 0.43 | 0.11 | 0.09 | 0.06 | |
| 0.074% | 0.172 | 0.86 | 0.172 | 0.086 | 0.021 | 0.017 | 0.011 | |
| 0.037% | 0.086 | 0.43 | 0.086 | 0.043 | 0.01 | 0.008 | 0.005 | |
| | 0.043% | 0.086% | 0.43% | 0.86% | 3.46% | 4.33% | 6.06% | Propionate |
| | | | | | | | | w/v |

The effect of the synergistic formulation over pathogens was evaluated to agronomical pests, specifically Botrytis cinerea and Pseudomonas syringae. In summary 100 μl of inoculum suspension was added into 96 wells in plates, using the following concentrations: 0.037-14.8% w/v acetate buffer and 0.043-17.31% w/v propionate buffer; 100 μl water (fungus) or culture medium (bacteria) and 10 μl of inoculum suspension at a final concentration of 1×10⁵ UFC/ml. An incubation of 24 hours at ±25° C. was performed, assays was made by triplicate. The antimicrobial effect was evaluated as follows: B. cinerea, a germinated conidia counting was made; and P. syringae, a minimal inhibitory concentration (MIC) was firstly made through presence/absence of turbidity, and from the concentrations in absence of turbidity 100 μl was taken to be transferred to a culture medium without the antimicrobial formulation and an incubation of 24 hours at room temperature was carried out to assay the minimal bactericide concentration (MBC).

TABLE 6
Percentage of inhibition to conidia germination of
B. cinerea produced by propionate, acetate and
a mixture of them, stated as adjusted mortality media

| | Concentration | Inhibition of |
|---|---|---|
| | (%, w/v) | germination (%) |
| Acetate (A) | 2.22 | 0.3 |
| | 5.17 | 7.4 |
| | 7.4 | 8.1 |
| Propionate (P) | 2.6 | 5.6 |
| | 6.06 | 18.1 |
| | 8.65 | 21.2 |
| Acetate + Propionate | 3.69 (A) + 4.33 (P) | 24.5 |
| | 5.17 (A) + 6.06 (P) | 40.1 |
| | 3.69 (A) + 8.65 (P) | 52 |
| | 5.17 (A) + 8.65 (P) | 53.4 |
| | 7.4 (A) + 8.65 (P) | 54.2 |

From table 6 it is noted that MBC to acetate buffer is 3.69% w/v while to propionate buffer such value was not possible to be determined. To the combination A+P, a proper effect (similar to against B. cinerea) was obtained to 2.95% w/v acetate buffer and 0.043-0.43% propionate buffer.

TABLE 7
Results of MIC (+) and MBC (−) against to P. syringae
of acetate buffer and propionate buffer by separate and mixed,
(−) no growth inhibition is noted, (+) growth inhibition is noted

| Acetate | | | | | | | | | |
|---|---|---|---|---|---|---|---|---|---|
| w/v | | | | | | | | | |
| 5.17% | ++ | ++ | ++ | ++ | ++ | ++ | ++ | ++ | |
| 3.69% | ++ | ++ | ++ | ++ | ++ | ++ | ++ | ++ | |
| 2.95% | + | ++ | ++ | ++ | ++ | ++ | ++ | ++ | |
| 0.74% | + | + | + | + | + | + | + | + | |
| 0.37% | + | + | + | + | + | + | + | + | |
| 0.074% | − | − | + | + | + | + | + | + | |
| 0.037% | − | − | + | + | + | + | + | + | |
| 0% | − | − | − | − | − | + | + | + | |
| | 0 | 0.043% | 0.086% | 0.43% | 0.86% | 3.46% | 4.33% | 6.06% | Propionate |
| | | | | | | | | | w/v |

### 2.2 Second Assay

For the assay, 10 μl of pathogenic bacteria culture (10⁸/ml) was exposed to 50 μl of synergistic mixtures for 30 minutes. Similar to example 5. At the end of this time the bacteria were diluted in peptonated water and stirred for 5 minutes, and from this mixture, 10 μl were serially diluted three times in TSB nutrient medium. Turbidity growth was recorded in the last dilution at 24 hours.

Bacteria of clinical, food and agricultural importance, among others (Table 8), were inhibited by the 1.72:1, 0.17 and 0.043 mixtures at the 15% and/or 35% concentration. Bacillus cereus was only inhibited at 35% concentration.

TABLE 8
Inhibition of the growth of pathogenic bacteria by synergists mixtures

| | Mixture | | | | | | | | |
|---|---|---|---|---|---|---|---|---|---|
| | 17.2:1 | | 1.72:1 | | 0.172:1 | | 0.043:1 | | Control |
| Concentración | 15% | 35% | 15% | 35% | 15% | 35% | 15% | 35% | 0% |
| P. aeruginosa | +++ | +++ | +++ | − | − | − | − | − | +++ |
| Klebsiella pneumoniae | +++ | +++ | +++ | − | − | − | − | − | +++ |
| Bacillus cereus | +++ | +++ | +++ | − | +++ | − | +++ | − | +++ |
| P. syringae | +++ | +++ | +++ | − | − | − | − | − | +++ |
| B. subtilis | +++ | − | − | − | − | − | − | − | +++ |

### 2.3 Acid Formulation

The efficacy of the synergistic proportions at acidic pH (1-4) was evaluated using the MEG and MBC assay. To determine MEG a final concentration of 10⁵ UFC/ml bacteria was inoculated at 96 multiwell plates containing 180 μl of different concentrations of disinfectant in TSB medium and the presence or absence of growing was observed to 20 minutes or 18-20 hrs of incubation, then to MBC 100 μl was taken from wells and re-inoculated in 5 ml of liquid nutritive TSB medium, finally tubes without growth was registered after 20 hrs. The range of disinfectant concentrations was 0.074-2.95% acetate buffer and 0.043-0.86% propionate buffer. These concentrations are obtained from a stock solution which is diluted with filtered water.

The results show that the proportions (17.2:1; 1.7:1; 0.17:1; 0.043:1) have a bactericidal effect on the pathogens E, coil and L. monocytogenes at acid pH, at 20 min of exposure. to the synergistic formulation.

TABLE 9
Efficacy of the synergistic proportions at acidic pH from MIC and MBC
assay. +++ Represents bactericidal effect, −−− Represents no effect

| | Mixture | | | | | | | | |
|---|---|---|---|---|---|---|---|---|---|
| | 17.2:1 | | 1.72:1 | | 0.86:1 | | 0.043:1 | | Control |
| Concentration | 3% | 5% | 3% | 5% | 3% | 5% | 3% | 5% | TSB |
| E. coli | −−− | +++ | +++ | +++ | +++ | +++ | −−− | +++ | −−− |
| L. monocytogenes | −−− | +++ | −−− | +++ | +++ | +++ | −−− | +++ | −−− |

### Example 3: Use of the Synergistic Formulation Against Microbial Biofilms

### 3.1: CDC Biofilm Reactor

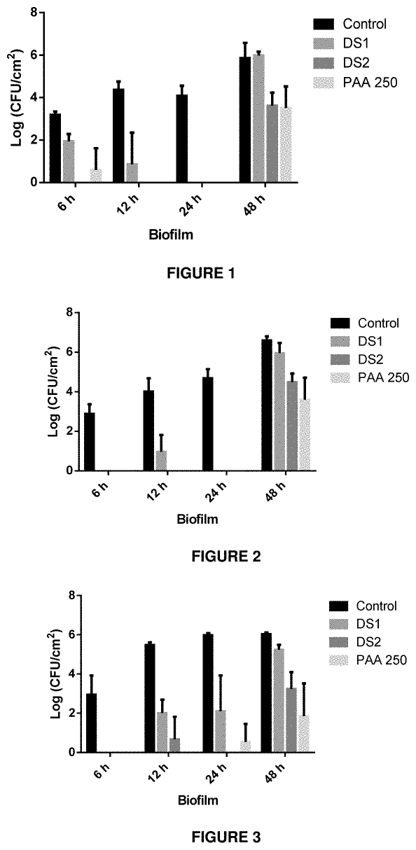

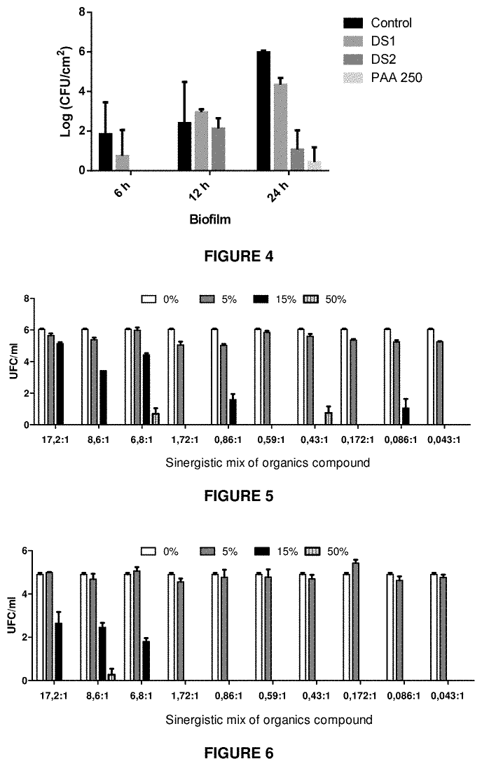

Tests on bacterial biofilms were also made to the pathogen strains, which were performed as follows: stainless steel coupons (type 316) were washed, installed on a support within a glass vessel and autoclaved at 121° C. by 20 minutes. To evaluate the disinfectant effect over a stablished biofilm to each separate bacteria, 3 ml of a concentration of 0.5 Mc Farland was inoculated within a vessel and then the same was incubated by 6, 12, 24 and 48 hours at 16° C. After each time of incubation supports containing coupons were removed and transferred to Falcon tubes of 50 ml containing the present synergistic disinfectant formulation at the following concentrations, DS1: 15% w/v (ratio acetate:propionate, 6.86:1) and DS2: 35% w/v (ratio acetate:propionate, 6.86:1). Peracetic acid was used (PAA) as control at concentrations of 250 ppm, the contact with the present synergistic disinfectant formulation or peracetic acid with supports containing biofilms was kept by 30 minutes. To perform a counting, an aliquot was taken, seeded into trypticase agar and incubated at 30° C. by 24 h, finally a counting of adhered cells was made. FIGS. 1 to 4 showing bacteria concentrations obtained to different times after 30 minutes of treatment using the above mentioned disinfectant formulations and peracetic acid as control to the following pathogens: L. monocytogenes ATCC 19115 (FIG. 1); E. coli ATCC 25932 (FIG. 2), Salmonella enterica ATCC 13076.L. (FIG. 3); and Staphylococcus aureus ATCC 6538P (FIG. 4).

The results as obtained showing as average the 4 strains of interest achieve a biofilm cell density between 10²-10³ UFC/cm² after 6 hours, 10⁴-10⁵ UFC/cm² after 12 hours, 10⁴-10⁶ UFC/cm² after 24 hours and 10⁵ UFC/cm² after 48 hours, evidencing an increase in the biofilm cell density measured as increases time of maturity.

Taken into account the tested concentrations to the synergistic disinfectant formulation, it is noted that a synergistic disinfectant formulation at a concentration of 15 w/v to the mixture of organic acids (Acetic acid+propionic acid, ratio acetate:propionate, 8,60:1) can reduce between 2-3 logarithm units (99-99.9%) of biofilm after 6 and 12 hours to all bacteria, excepting S. aureus. While such formulation at a concentration of 35% w/v to the mixture of organic acids (Acetic acid+propionic acid, ratio acetate:propionate, 6.86:1) can eliminate 4 logarithm units (99.99%) of biofilm excepting S. aureus. In case of peracetic acid, biofilms were completely reduced.

To 24-hours aged biofilms, the present synergistic disinfectant formulation at a concentration of 15% w/v to the mixture of organic acids (Acetic acid+propionic acid, ratio acetate:propionate; 6.86:1) can eliminate only the L. monocytogenes and E. coli biofilms while the same can reduce 99% and 99.9% S. aureus and S. enterica biofilms, respectively. Peracetic acid can fully eliminate all the bacteria biofilms. To biofilms 48-hours aged, the present synergistic disinfectant formulation can reduce between 2-3 log units (99-99.9%) the bacteria biofilms, having an effect similar to 250 ppm peracetic acid.

### 3.2 Control of 24 h Bacterial Biofilms on Polypropylene Multi-Well Cell Culture Plates

To explore different synergistic mixtures of acetate and propionate solutions on biofilms of pathogenic bacteria, gram negative bacteria E. coli ATCC 25932 and Salmonella enterica ATCC 13076 and gram-positive L. monocytogenes ATCC 19115; Staphylococcus aureus ATCC 6538P were used. Synergistic mixtures are presented in Table 10.

To demonstrate this, cultures grown for 24 h in TSB (room temperature, 120 rpm) were diluted to 1-5×10⁶ cells/ml. In 96-well plates (flat bottom) containing 180 ul of TSB, 20 ul of this dilution was inoculated and incubated for 24 hours at 14° C., 120 rpm. After this time, the liquid medium is removed and each well is washed twice with 250 ul of sterile water. 250 ul of synergist disinfectant mixture (Table 10) in concentrations 0, 5, 15 and 50% was added to each well, and left to act for 30 minutes. After that time, the disinfectant was removed and the wells were washed twice with 300 ul of peptone water, leaving the last wash to act for 10 min. (neutralizing) This water is removed and the biofilm adhered to the surface of each well is resuspended in 200 ul of peptone water, by successive shaking and pipetting. 10 ul are deposited in 190 ul of TSB (in another 96-well plate) to evaluate the biocidal effect at 24 and 48 h. 100 ul are serially diluted in tubes containing 900 ul of water, counting by microdroplet.

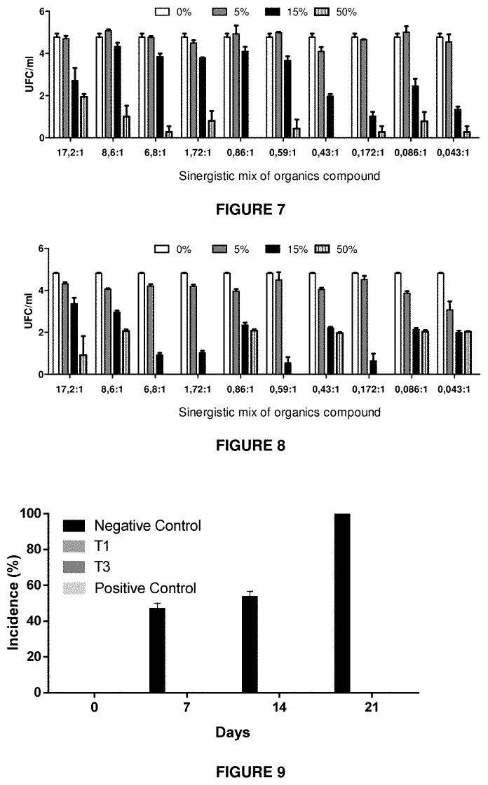

Synergistic mixtures reduce the count of gram-negative bacteria such as E. coli (FIG. 5), and S. enterica (FIG. 6) and gram-positive bacteria such as S. aureus (FIG. 7) and L. monocytogenes (FIG. 8). A significant decrease in the bacterial count was observed with increasing concentration of the mixtures. The mixtures with concentrations of 15% and from the ratio 17.2:1 to those with lower proportions of acetate were effective reducing more than 99.999%. 50% mixes were always effective. The same concentrations showed a biocidal effect on these bacteria (Table 10 for E. coli, S. enterica, S. aureus and L. monocytogenes).

TABLE 10
Biocidal effect of synergistic mixtures on different pathogenic bacteria
(E. coli ATCC 25932, Salmonella enterica ATCC 13076, Staphylococcus aureus
ATCC 6538P y Listeria monocytogenes ATCC 19115) at 24 h/48 h. +
represents growth, and − absence of turbidity..

| | E. coli | | | S. enterica | | | S. aureus | | | L. monocytogenes | | |
|---|---|---|---|---|---|---|---|---|---|---|---|---|
| | 25932 | | | 13076 | | | 6538P | | | 19115 | | |
| Mixtures | 5% | 15% | 50% | 5% | 15% | 50% | 5% | 15% | 50% | 5% | 15% | 50% |
| 17.2:1 | +/+ | +/+ | − | +/+ | +/+ | − | +/+ | +/+ | − | +/+ | +/+ | − |
| 8.6:1 | +/+ | −/+ | − | +/+ | +/+ | − | +/+ | +/+ | − | +/+ | +/+ | − |
| 6.8:1 | +/+ | −/+ | − | +/+ | −/+ | − | +/+ | +/+ | − | +/+ | − | − |
| 1.72:1 | +/+ | − | − | +/+ | − | − | +/+ | +/+ | − | +/+ | − | − |
| 0.86:1 | +/+ | −/+ | − | +/+ | − | − | +/+ | +/+ | − | +/+ | +/+ | − |
| 0.59:1 | +/+ | − | − | +/+ | − | − | +/+ | +/+ | − | +/+ | − | − |
| 0.43:1 | +/+ | − | − | +/+ | − | − | +/+ | − | − | +/+ | − | − |
| 0.172:1 | +/+ | − | − | +/+ | − | − | +/+ | − | − | +/+ | − | − |
| 0.086:1 | +/+ | − | − | +/+ | − | − | +/+ | − | − | +/+ | − | − |
| 0.043:1 | +/+ | − | − | +/+ | − | − | +/+ | − | − | +/+ | − | − |

### Example 4: Use of the Formulation Over Foods

### 4.1 Assays in Blueberry, Tangerine and Plum Fruits

Fruits having similar, size, form and color and having no visual damage by fungi were selected. To perform assays a 1-2 mm deep wound was made on the selected fruit by a needle and 10⁵ UFC/ml B. cinerea and Penicillium spp. (to different samples, independent test) were inoculated through the same wound under sterility conditions, fruits was not disinfected prior to the inoculation procedures. Assays was made by triplicate on 6 units submitted to visual inspection during the treatment, which were equidistantly located therebetween in Petri plates of 100×15 mm and exposed to a disinfectant treatment (Different proportions and concentrations) spraying on the fruit 0.2 ml of the different synergistic proportion of the formulation, finally an incubation at 4° C. was allowed.

Four acetate/propionate mixtures (proportions) were tested, 17.2:1, 6.86:1, 1.72:1 and 0.043:1; for each of them two or three concentrations were tested. Treatments: Treatment A/Treatment 1: 3.38% w/v mixture acetate/propionate buffers. Treatment B/Treatment 2: 11.25% w/v mixture acetate/propionate buffers. Treatment C: 25% w/v mixture acetate/propionate buffers. Treatment D/Negative Control: Fruits with wound immersed in water. Treatment E/Positive Control: Fruits plus benzalkonium chloride. Treatment F/Absolute Control: Water and wound-free Fruits, also designated as control to fruit quality.

Assays were evaluated to days 0 (control), 7, 14 and 21, a counting of fruits showing pathogen signs was made to determined incidence. Each assay was independently performed to each fruit, and a totally random factorial design was used as experimental design to each assay.

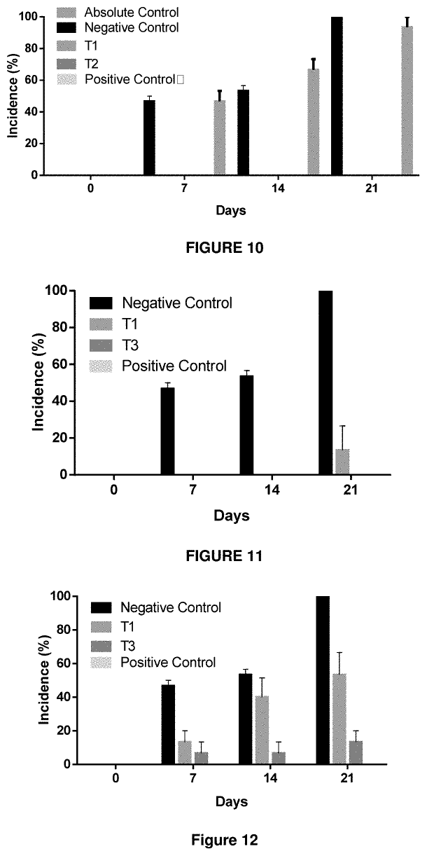

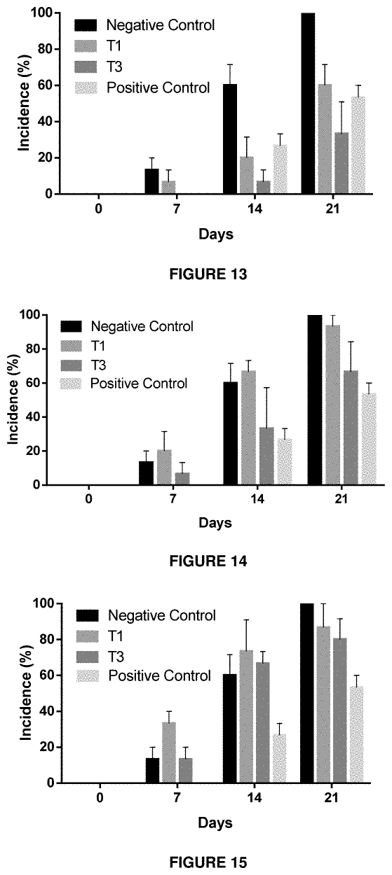

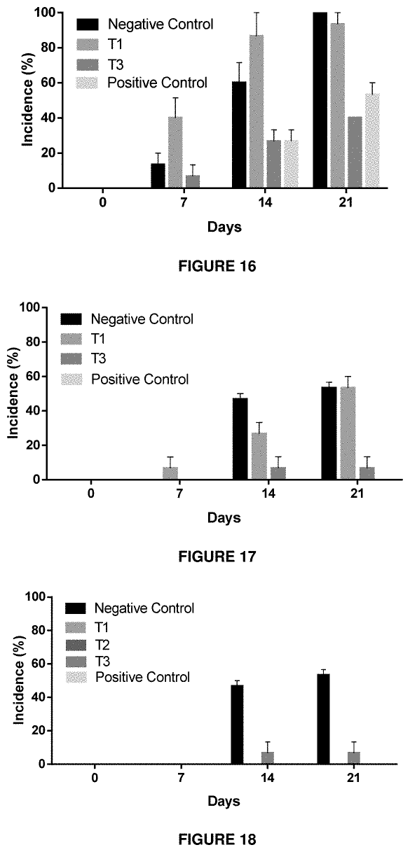

To blueberry infected with B. cinerea fruits was noted that controls achieve a 50% of disease incidence at the 14 days, while treatments 1 y 2 inhibit a disease incidence at least for a while of 21 days, achieving the same results that benzalkonium chloride (commercial disinfectant, positive control). Further, no difference was confirmed between absolute control (directly purchased fruits) and negative control to which water was added to keep the moisture that disinfectant causes on the fruit. These results show that the effect is linked to the features of the used disinfectant but not to the fact that it can be moistened and kept on the fruit the same trend is observed in all the portions tested (FIG. 9, FIG. 10, FIG. 11, FIG. 12); and also, when they were tested against Penicillium spp., however, there is a difference in the relationship between the fungi and the fruit (FIG. 13, FIG. 14, FIG. 15, FIG. 16).

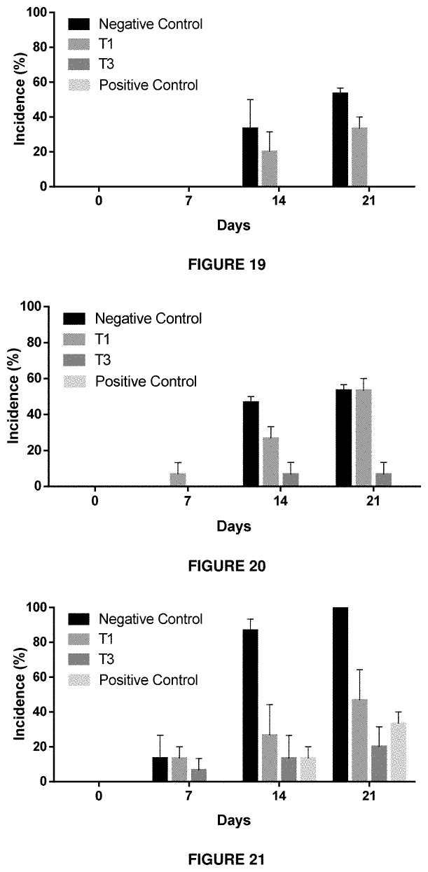

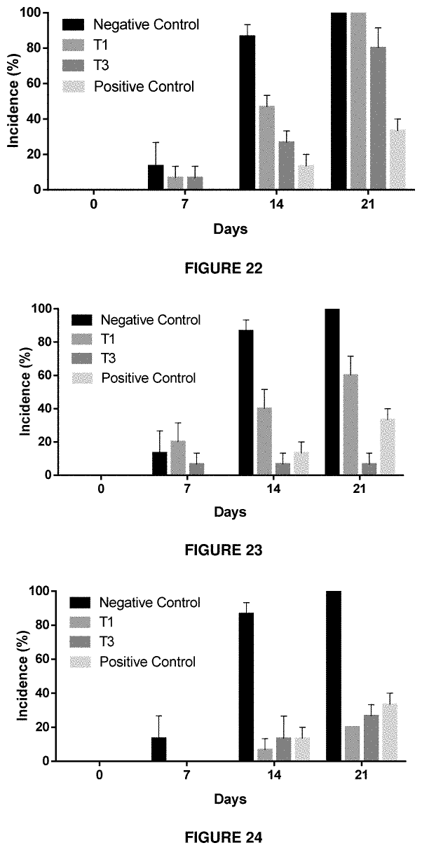

To 21-days aged fruits, treatment inhibits a pathogen disease up to 21 days while treatment 2 inhibits a pathogen disease at least 28 days. Positive control inhibits incidence by 42 days, however, treatments 1 and 2 show a similar effect than positive control. In the case of plum, all the proportions at 25% have a controlling effect, however the difference between the pathogen and the proportions is observed, for example, for plums infected with B. cinerea (FIG. 17, FIG. 18, FIG. 19, FIG. 20), the most effective ratio is 6.86:1, but for plums infected with Penicillium spp. The ratio 0.043:1 is the one that shows the greatest effect (FIG. 21, FIG. 22, FIG. 23, FIG. 24).

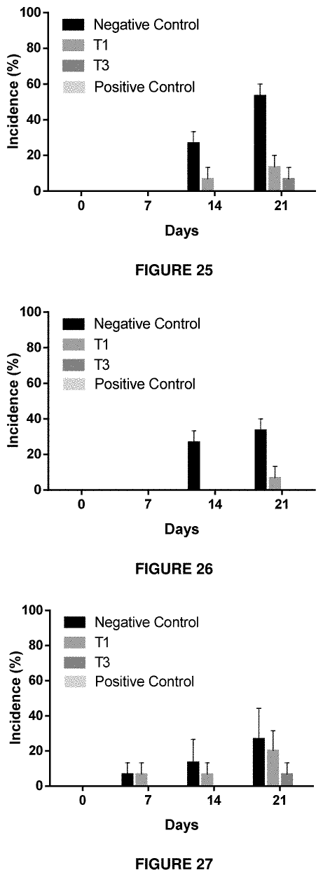

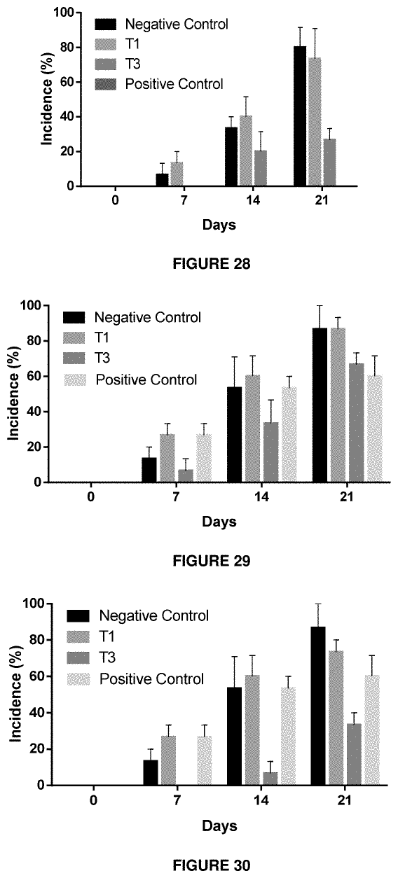

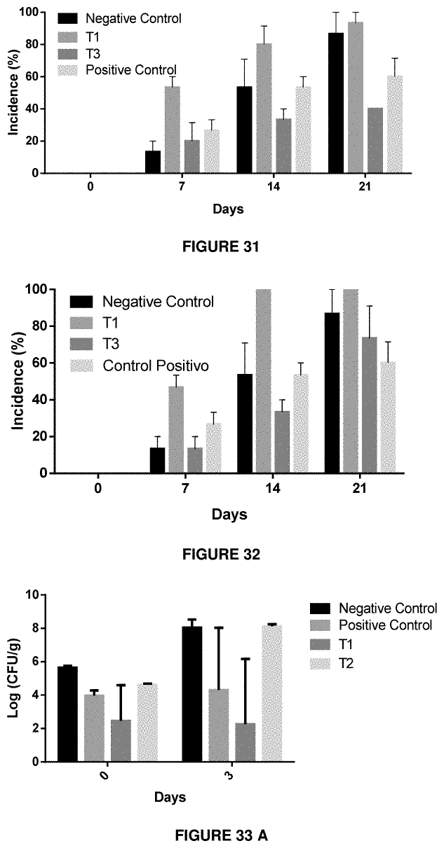

In the case of mandarin orange infected with Botrytis cinerea, it is observed that the fungicidal effect increases with concentration, however, the proportions 17.2:1 and 6.86:1 are those that maintain this effect for 21 days, instead the proportions 1.72:1 and 0.043 lose effect after 14 days (FIG. 25, FIG. 26, FIG. 27, FIG. 28). For mandarins infected with Penicillium, it is observed that none of the proportions have a controlling effect at 3.38%, however, at 25% they present a lower microbial load than the positive control (FIG. 29, FIG. 30, FIG. 31, FIG. 32).

From this trial it could be concluded that the different kind of fruit and fungi specie had a specific response to the proportions and concentrations of the formulation tested, possibly due to the affinity for the acids that predominate in the mixture.

### 4.2 Assays on Meat

Assays to evaluate antimicrobial effect on the surface of beef, chicken meat and salmon meat was performed. To avoid the influence of external factors on the experimental results, the above mentioned 3 types of meat were purchased in a same market point and were chosen no preservative treated meat. The growth of bacteria Escherichia coli was allowed in a TSB culture medium by 24 h at 35° C.

Disinfecting effect on a total mesophyll aerobic and enterobacteria microorganism counting was evaluated to initial non inoculated (E. coli) meat. Also, a concentration of 10¹ UFC/g of E. coli was inoculated and then the disinfectant effect was evaluated on such pathogen microorganism counting.

Meat (beef, chicken and salmon) was purchased the same date in which the assay was arranged and was kept under storing conditions at 4° C. up to using the same. Prior to initiating assays pieces of 5 grams, pieces were randomly selected to each treatment, and a) pieces were independently inoculated with 0.2 ml of culture comprising the above mentioned bacteria by separate and 2 ml was applied to the treatments to be defined below or b) pieces were only chopped and 2 ml of treatment was applied:

For a mixture acetate buffer/propionate buffer (ratio acetate:propionate; 6.86:1)—T1: Synergistic Disinfectant Formulation comprising 3.38% w/v—T2: Synergistic Disinfectant Formulation comprising 0.67% w/v. For a mixture acetate buffer/propionate buffer (ratio acetate:propionate; 0.043:1)—T3: Synergistic Disinfectant Formulation comprising 3.38% w/v—T7: Synergistic Disinfectant Formulation comprising 25% w/v. For a mixture acetate buffer/propionate buffer (ratio acetate:propionate; 0.172:1)—T4: Synergistic Disinfectant Formulation comprising 3.38% w/v—T8: Synergistic Disinfectant Formulation comprising 25% w/v. For a mixture acetate buffer/propionate buffer (ratio acetate:propionate; 1.72:1)—T5: Synergistic Disinfectant Formulation comprising 3.38% w/v—T9: Synergistic Disinfectant Formulation comprising 25% w/v. For a mixture acetate buffer/propionate buffer (ratio acetate:propionate; 17.2:1)—T6: Synergistic Disinfectant Formulation comprising 3.38% w/v—T10: Synergistic Disinfectant Formulation comprising 25% w/v—Negative Control (CN): Water—Positive Control (CP): 2.5% Sodium Acetate.

Meat pieces (inoculated or non-inoculated) with its respective treatments was transferred to tubes of 50 mL under conditions of sterility and stored at 4° C. up to its final evaluation. Assays were made by triplicate.

Antimicrobial activity evaluation was performed to 0, 3, 8 and 12 post-treatment days. Thus, meat pieces of 5 gr were submerged in 10 mL TSB culture medium, each tube was sonicated 3 times by 1 minute at each time and submitted to stirring in each interval of time. A seriated dilution of samples was made and the same were seeded in TSA plates at 100% or XLD selective medium by a rake method. Colonies were counted at 24 and 48 hours, depending the medium to be used to the counting.

### 4.2.1 Assay to Evaluate Disinfectant Effect in Meats Having Microbial Loading

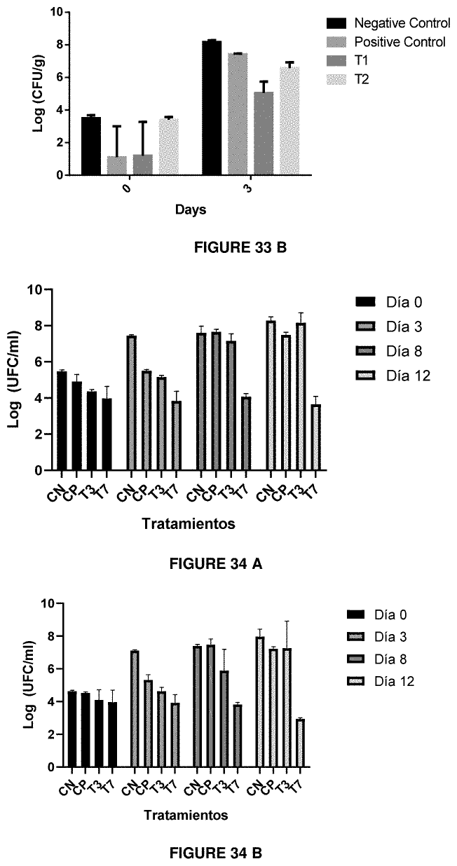

To the disinfectant effect over the microorganisms present in non-inoculated meat, it is observed that the following: For ratio 6.86:1—chicken, T1 shows higher effects compared to positive control in the total counting of mesophyll aerobics (FIG. 33A) as well to the selective enterobacteria medium (FIG. 33B), and said effect is kept after 3 days. Also, T1 having an effect from the first contact (day 0), suggesting a washing disinfectant use. At the day 3, T1 reduces in 4 log units the counting of mesophyll aerobics, which is equivalent to 99.99%. This result after compared against to the positive control is superior in 2 log units. To enterobacteria, this trend is similar since T1 reduces in 3 log units the enterobacteria counting and is higher in 2 log units to the positive control. While to T2 a reduction of 1.5 log units compared to the negative control is observed.

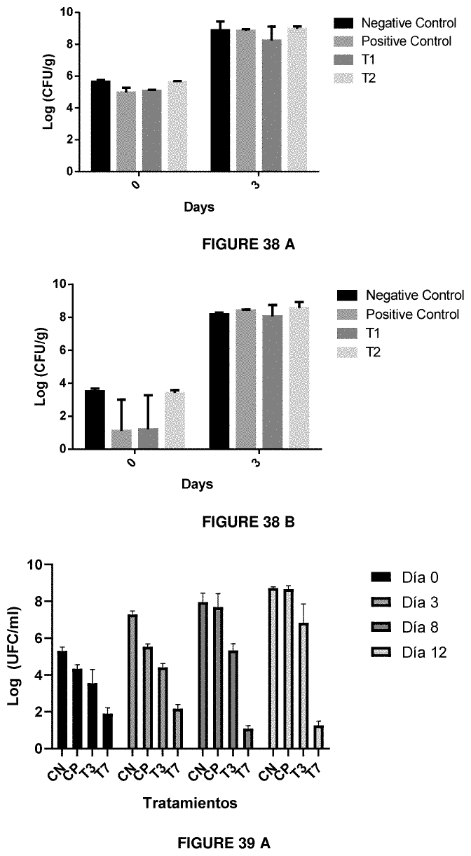

To meat, it was observed that T1 reduces 0-5 log units (FIG. 38A) the total counting of mesophyll aerobics. A same range is kept after 3 days compared to the control. To the counting of enterobacteria (FIG. 38B), T1 reduces in 2 log units at day 0, this difference cannot be kept after 3 days.

To salmon meat, the total counting of mesophyll aerobics (FIG. 43A) there is an antimicrobial effect to both concentrations, T1 and T2, in day 0. To T1 it is observed a reduction of 1.5 log units while T2 is 1 log unit, being the first treatment equivalent to the positive control. A same trend is kept after 3 days. Respect to the counting of enterobacteria (FIG. 43B) it is noted that T1 reduces over 2 log units compared to negative control, such difference increases in 3 log units (99.9%) after 3 days. To T2, there is no difference in the counting at day 0, however, the disinfectant has an effect at day 3, reducing in 2 log units the growth of enterobacteria in salmon meat.

To the 3 types of meats a similar trend is present, in which T1 a reduction between 99-99.99% is achieved to the total counting of mesophyll aerobics and enterobacteria naturally in meat at the commercialization time.

For 0.043:1 ratio in chicken (FIG. 34A) using the T3 concentration achieves a decrease of between 1-2 Log (90-99%) the mesophilic aerobic count between days 0-8 and between 2-3 log (99 99.9%) the count of Enterobacteriaceae (FIG. 34B) on days 3 and 8. The T7 concentration eliminates 99.9% of the aerobic mesophilic and enterobacteria count from day 0, maintaining the same efficacy until day 12.

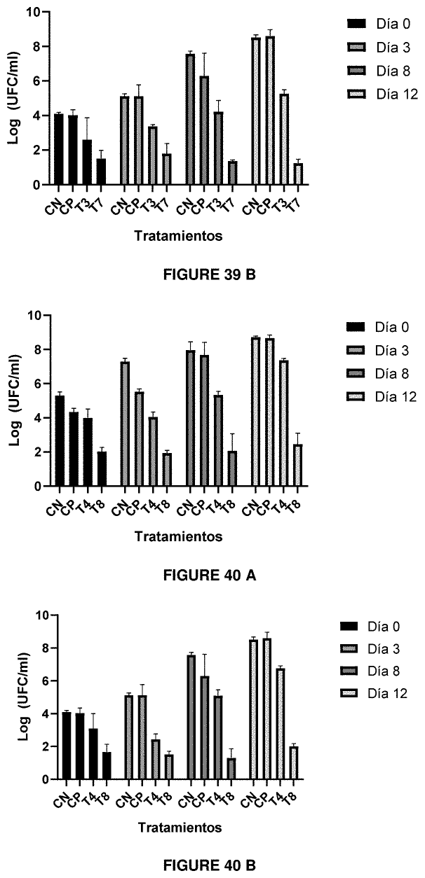

In beef (FIG. 39A), the T3 concentration reduces the count of mesophilic aerobes between 1-3 log (90-99.9%) between day 0-12 and between 1-4 log (90-99.99%) the count of enterobacteria on days 0-12, obtaining the greatest decrease in the mesophilic aerobic count on day 3 and for enterobacteria (FIG. 39B) on days 8 and 12. The T7 concentration on day 0 manages to decrease the count of mesophilic and enterobacterial aerobes by 3 Log (99.9%), reaching up to 7 log (99.99999%) on day 12.

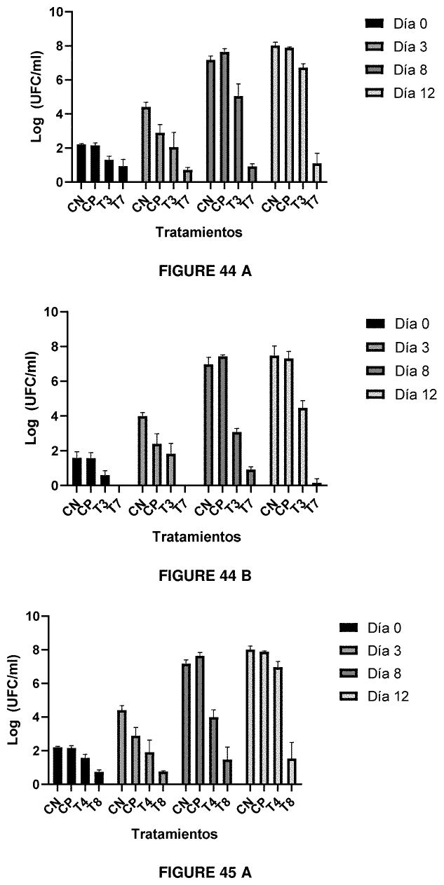

In salmon (FIG. 44A), the T3 concentration achieves a reduction of between 1-2 log (90-99%) the mesophilic aerobic count between days 3-12 and between 1-3 log (99-99.9%) the count of Enterobacteriaceae (FIG. 44B) on days 8 and 12. The T7 concentration achieves a reduction between 1-7 Log the count of mesophilic aerobes and enterobacteria, with days 8 and 12 being the most efficient.

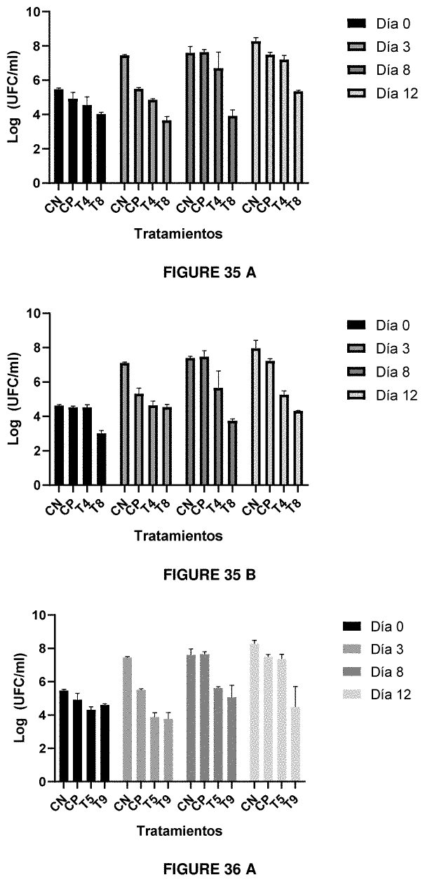

For 0.172:1 ratio in chicken (FIG. 35A) using the T4 concentration manages to decrease between 1-2 Log (90-99%) the mesophilic aerobic count between days 0-8 and enterobacteria (FIG. 35B) between days 0-12. The T8 concentration manages to decrease between 1-3 Log (90-99.9%) the count of mesophilic aerobes and Enterobacteriaceae between days 0-12.

In beef (FIG. 40A), the T4 concentration achieves a reduction between 1-3 Log (90-99.9%) the count of mesophilic aerobic bacteria and enterobacteria (FIG. 40B), being the highest efficacy on days 3 and 8. The T8 concentration achieves a reduction between 3-6 log the count of aerobic mesophiles and Enterobacteriaceae, with the highest efficacy on days 8 and 12.

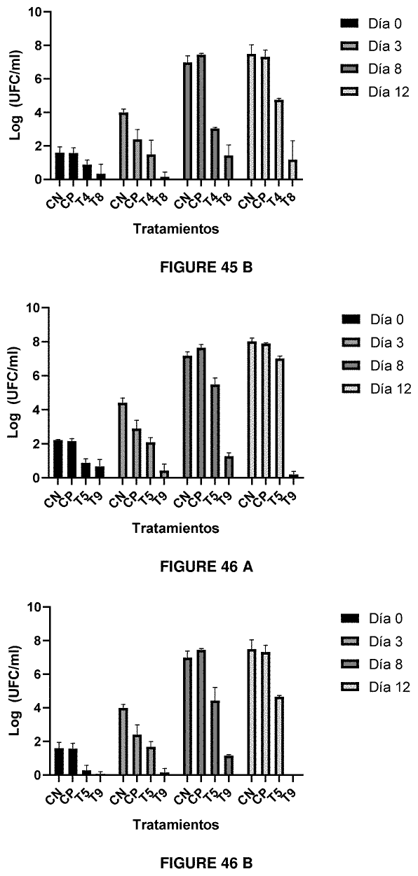

In salmon (FIG. 45A), the T4 concentration achieves a reduction between 1-3 log (90-99.9%) the count of mesophilic aerobic bacteria and enterobacteria (FIG. 45B) between days 3-12, with the highest efficacy being on days 3 and 8. The T8 concentration achieves a reduction between 1-6 Log the count of mesophilic aerobes and Enterobacteriaceae, with days 8 and 12 being the most efficient.

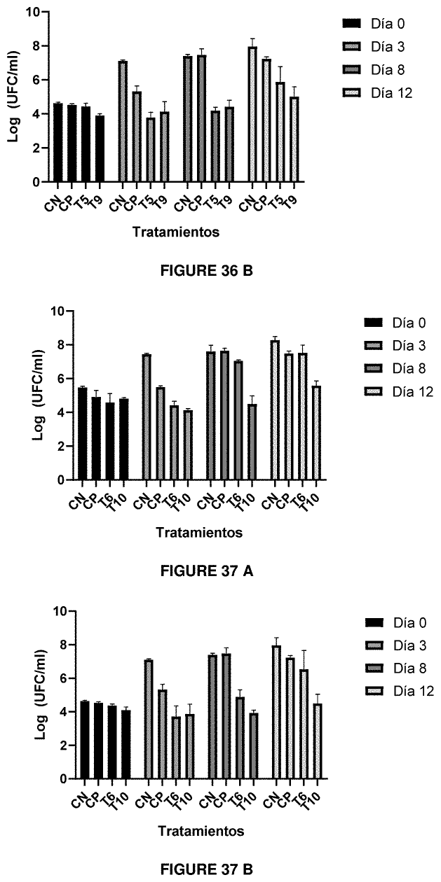

For 1.7:1 ratio, in chicken (FIG. 36A) using the T5 concentration achieves a reduction between 1-3 Log (90-99.9%) the count of mesophilic aerobes between days 0-8 and of Enterobacteriaceae (FIG. 36B) between days 0-12, being the highest effect at day 3 and 8, respectively. The T9 concentration reduces the mesophilic aerobic count by 2-3 log and the Enterobacteriaceae count by 2 log between days 3-12.

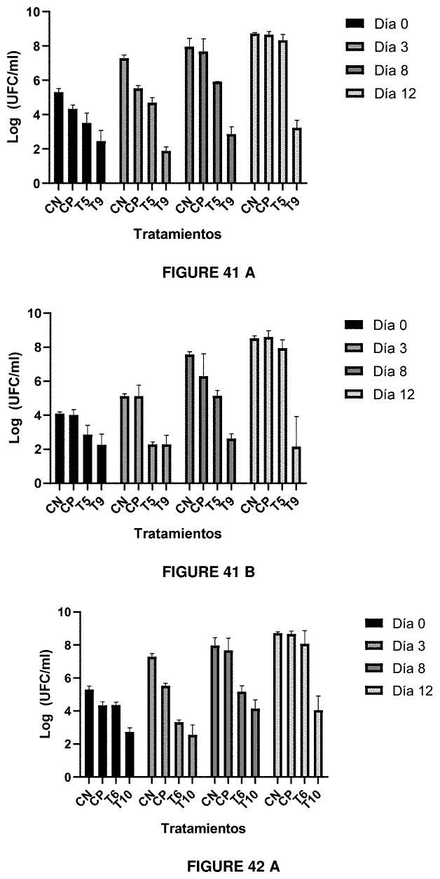

In beef (FIG. 41A), the T5 concentration achieves a reduction of between 1-3 Log the count of mesophilic aerobes and enterobacteria (FIG. 41B) between days 1-8, with days 3 and 8 being the most efficient. The T9 concentration achieves a reduction between 2-5 log and between 1-6 log the count of mesophilic aerobes and enterobacteria, respectively between days 1-12, with the highest efficacy being on days 8 and 12.

In salmon (FIG. 46A), the T5 concentration achieves a reduction of between 1-2 log (90-99%) the mesophilic aerobic count between days 0-12 and between 1-3 log (99-99.9%) the count of Enterobacteriaceae (FIG. 46B) between days 0 and 12. The T9 concentration achieves a reduction of between 1-7 Log the count of mesophilic aerobes and Enterobacteriaceae, with days 8 and 12 being the most efficient.

For 17.2:1 ratio in chicken (FIG. 37A) using the T6 concentration achieves a reduction in the count of mesophilic aerobes and enterobacteria (FIG. 37B) between 2-3 logs on days 3 and 8. The T10 concentration achieves a reduction of mesophilic and enterobacterial aerobes between 2-3 logs between days 3-12.

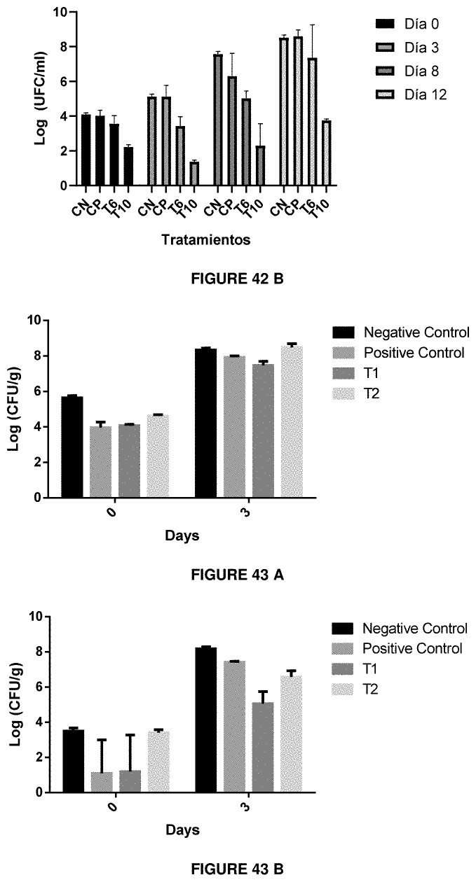

In beef (FIG. 42A), the T6 concentration achieves a reduction of the mesophilic aerobic count between 2-3 log and the Enterobacteria (FIG. 42B) count between 1-2 log on days 3 and 8. The T10 concentration achieves a reduction of the mesophilic aerobic count and Enterobacteriaceae between 3-5 log between days 3-12.

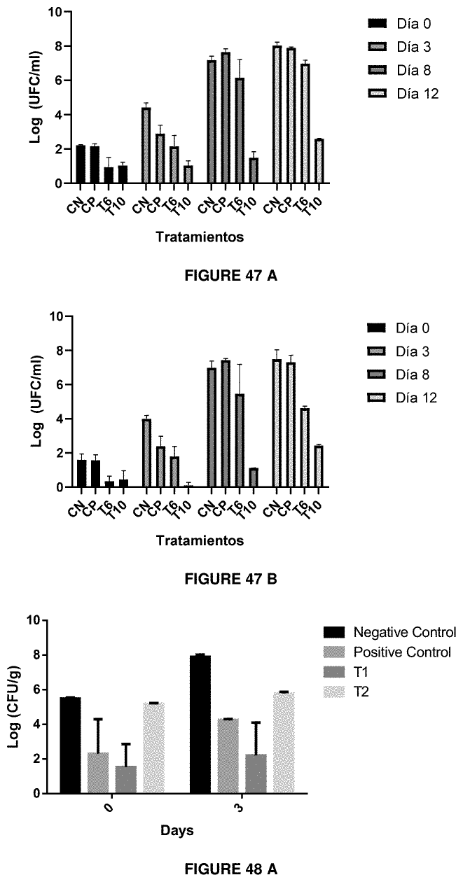

In salmon (FIG. 47A), the T6 concentration achieves a reduction of the aerobic mesophilic and enterobacterial (FIG. 47B) count between 1-2 log between days 0-12, being more effective on day 3. The T10 concentration achieves a reduction of between 1-5 log the aerobic count mesophiles and enterobacteria between days 0-12.

For the proportions 0.043; 0.17; 1.7 and 17.2, it can be seen that the sanitizing efficacy can be observed from day 3, regardless of the concentration used, which indicates that these proportions tend to increase the shelf life of the food, rather than having a washing effect.

The concentration at 25% w/v was more effective (2-3 log more reduction) in meat and salmon than in chicken, which may be influenced by the type of composition of each feed. Also, in meat at days 0 and 12 it can be seen that those proportions that have a greater amount of propionate, have a greater efficiency than those where there is a greater presence of acetate.

The results show that the proportions (0.043:1; 0.17:1; 1.7:1 and 17.2:1) show greater efficacy than the positive control used (sodium acetate), which had aerobic mesophilic and enterobacterial counts close to the negative control (only water) for the different meats, during test days. From FIGS. 34-47, it is noted that all the synergistic ratios show a good efficacy reducing the count of mesophilic aerobes and enterobacteria from day 3. This effect is remarked in beef and salmon meat.

### 4.2.2 Assay of Evaluation to the Disinfectant Effect in Human Pathogen-Inoculated Meats (10¹ Cells/g Meat).

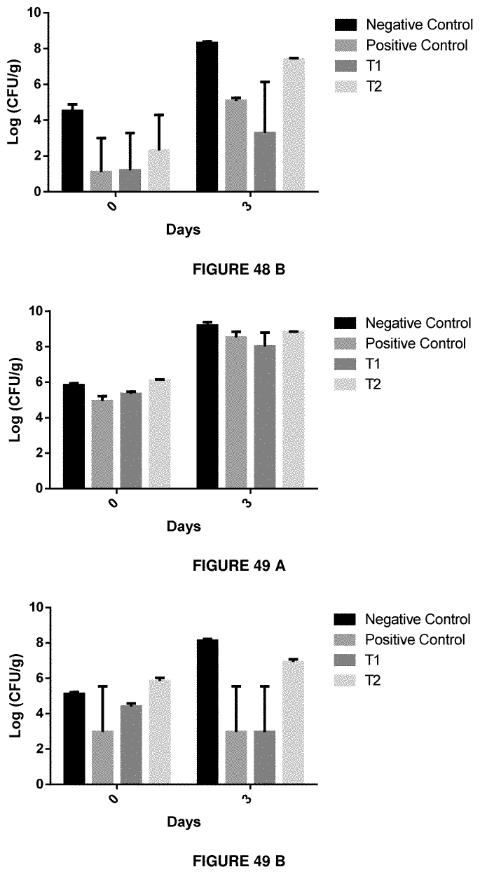

To inoculated meat with a concentration of 10¹ UFC/g of E. coli, it is observed in salmon meat that T1 reduces in 3 log units the total counting of mesophyll aerobics at day 0, being the log unit lower the positive control. To T2 at day 3, there is a reduction of 2 log units of the counting compared to the negative control (FIG. 48).

To the counting of E. coli over salmon meat (FIG. 48B) it is observed a reduction of 2 log units to T1 compared to the negative control at day 0, and this effect can be kept after 3 days, being the effect higher to positive control. To T2 only 1 log unit can be reduced at day 3. In beef (FIG. 49) it is noted a reduction of 1 log unit to T1, and this effect is kept after day 3. In the counting to E. coli (FIG. 11B) it is noted an effect of reduction 1 log unit only at day 0 to T1, which increases to 5 log units at day 3, achieving the effect of positive control.

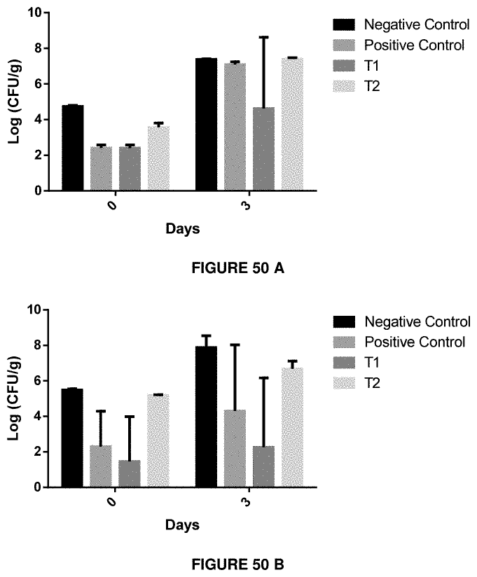

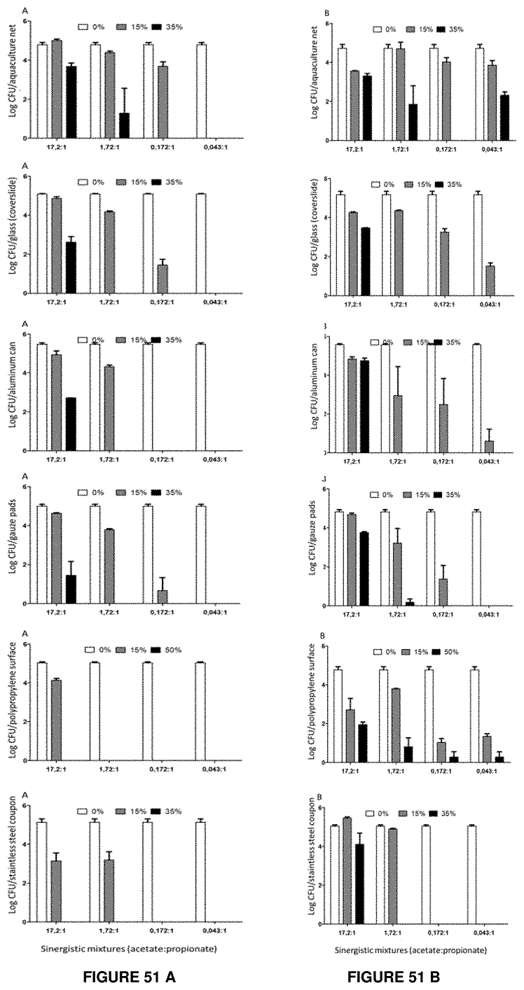

In chicken meat inoculated with E. coli (FIG. 50), the total counting of mesophyll aerobics shows a reduction of 4 log units when treated with T1 either at day 0 or day 3, obtaining a reduction of 2 log units' lower positive control.

### Example 5: Use of the Formulation on Different Surfaces

Four acetate/propionate mixtures (proportions) were tested; 17.2:1, 1.7:1, 0.17:1 and 0.043:1. For each of them two concentrations were tested (15% and 35%) on bacteria faced surfaces, 10 μl of old cultures (10⁸ cel/ml) was deposited on the surface such us glass (coverslide), sterile gauze pads, aluminum drink can, knotless raschel net, stainless steel coupon, polypropylene (Example 3). The size of the surfaces varies between 1 cm² coupons and cans, 2 cm² net, 2.5 cm² gauze. This test was based on “Quantitative method for evaluating bactericidal activity of Microbicides used on hard non-porous surfaces (OECD 2013). In short, 50 μl of acetate/propionate mixture is allowed to act on the inoculum. And then the bacteria are recovered by resuspending them from the surfaces in 10 ml of water and shaking for 30 sec.

Efficacy tests of the mixtures were performed on E. coli (FIG. 51A) and S. aureus (FIG. 51 B) bacteria fixed on different surfaces, including aquaculture nets, glass, aluminium can, gauze pads, polypropylene and stainless steel surfaces. Differences were observed between Gram negative and positive bacteria, the latter more persistent after treatment; however, 0.172:1 was always efficient at 35% on both types of bacteria (99.99% reduction) and all surfaces tested. Mixtures 1.72:1, 0.172:1 and 0.043:1 at 35% reduced the bacterial count between 99.9-99.999% on the different surfaces.

1. A synergistic bactericide and bacteriostatic organic sanitizing/disinfectant/cleaning formulation comprising a mixture of acetic acid and propionic acid or salts thereof at a ratio acetate:propionate from 4:100 to 20:1.

2. The synergistic bactericide and bacteriostatic organic sanitizing/disinfectant/cleaning formulation of claim 1 having a ratio acetate:propionate selected from 4.3:100, 8.6:100, 17.2:100, 43:100, 59:100, 86:100, 1.72:1, 4.30:1, 6.86:1, 8.6:1, 17.2:1 and 34.30.

3. The synergistic bactericide and bacteriostatic organic sanitizing/disinfectant/cleaning formulation of claim 1 having a concentration of such mixture of acetic acid and propionic acid or salts thereof in a range of 0.5 to 50% w/v in the formulation.

4. The synergistic bactericide and bacteriostatic organic sanitizing/disinfectant/cleaning formulation of claim 3 wherein such concentration of such mixture of acetic acid and propionic acid or salts thereof in the formulation, is selected from 0.67%, 3%, 3.38%, 5%, 11.25%, 15%, 17.14%, 20%, 25%, 34.29%, 35%, or 50% w/v.

5. The synergistic bactericide and bacteriostatic organic sanitizing/disinfectant/cleaning formulation of claim 3 wherein such concentration of such mixture of acetic acid and propionic acid or salts thereof in the formulation is 0.67% w/v.

6. The synergistic bactericide and bacteriostatic organic sanitizing/disinfectant/cleaning formulation of claim 3 wherein such concentration of such mixture of acetic acid and propionic acid or salts thereof in the formulation is 3% w/v.

7. The synergistic bactericide and bacteriostatic organic sanitizing/disinfectant/cleaning formulation of claim 3 wherein such concentration of such mixture of acetic acid and propionic acid or salts thereof in the formulation is 3.38% w/v.

8. The synergistic bactericide and bacteriostatic organic sanitizing/disinfectant/cleaning formulation of claim 3 wherein such concentration of such mixture of acetic acid and propionic acid or salts thereof in the formulation is 5% w/v.

9. The synergistic bactericide and bacteriostatic organic sanitizing/disinfectant/cleaning formulation of claim 3 wherein such concentration of such mixture of acetic acid and propionic acid or salts thereof in the formulation is 11.25% w/v.

10. The synergistic bactericide and bacteriostatic organic sanitizing/disinfectant/cleaning formulation of claim 3 wherein such concentration of such mixture of acetic acid and propionic acid or salts thereof in the formulation is 15% w/v.

11. The synergistic bactericide and bacteriostatic organic sanitizing/disinfectant/cleaning formulation of claim 3 wherein such concentration of such mixture of acetic acid and propionic acid or salts thereof in the formulation is 17.14% w/v.

12. The synergistic bactericide and bacteriostatic organic sanitizing/disinfectant/cleaning formulation of claim 3 wherein such concentration of such mixture of acetic acid and propionic acid or salts thereof in the formulation is 20% w/v.

13. The synergistic bactericide and bacteriostatic organic sanitizing/disinfectant/cleaning formulation of claim 3 wherein such concentration of such mixture of acetic acid and propionic acid or salts thereof in the formulation is 25% w/v n.

14. The synergistic bactericide and bacteriostatic organic sanitizing/disinfectant/cleaning formulation of claim 3 wherein such concentration of such mixture of acetic acid and propionic acid or salts thereof in the formulation is 34.29% w/v.

15. The synergistic bactericide and bacteriostatic organic sanitizing/disinfectant/cleaning formulation of claim 3 wherein such concentration of such mixture of acetic acid and propionic acid or salts thereof in the formulation is 35% w/v.

16. The synergistic bactericide and bacteriostatic organic sanitizing/disinfectant/cleaning formulation of claim 3 wherein such concentration of such mixture of acetic acid and propionic acid or salts thereof in the formulation is 50% w/v.

17. The synergistic bactericide and bacteriostatic organic sanitizing/disinfectant/cleaning formulation of claim 1 having pH from 4 to 6.

18. Use of the synergistic organic sanitizing/disinfectant/cleaning formulation of claim 1, useful to sanitizing and sterilization food as well surfaces.

19. The use of the synergistic organic sanitizing/disinfectant/cleaning formulation of claim 6, useful as a Generally Recognized as Safe Compound (GRAS).

20. The use of claim 18 wherein such food is selected from harvested or fresh fruits and vegetables including blueberry, tangerine and plum, and meat including beef, chicken meat and salmon meat.

21. The use of claim 18 wherein such hard surfaces is selected from domestic surfaces including floors and furniture, industrial surfaces; and hospital surfaces including medical or dental tools and equipment surfaces.

22. Use of the synergistic organic sanitizing/disinfectant/cleaning formulation of claim 1, useful to combating and/or eliminating microorganisms including Listeria monocytogenes, Salmonella enterica, Escherichia coli, Staphylococcus aureus, Botrytis cinerea, Pseudomonas aeruginosa, Klebsiella pneumoniae, Bacillus cereus, Bacillus subtilis o Pseudomonas syringae or its biofilms.

23. The formulation of claim 1 further comprising natural or synthetic fragrances, natural or synthetic colorant agents, natural or synthetic surfactant, natural or synthetic organic additional active additives, natural or synthetic emulsifiers or natural or synthetic thickeners.

24. The bactericide and bacteriostatic organic sanitizing/disinfectant/cleaning composition of claim 1 which can be applied by direct contact, cold immersion or glazed, spray, fogging, aspersion, or immersion.

25. The synergistic bactericide and bacteriostatic organic sanitizing/disinfectant/cleaning formulation of claim 1 which is freeze-dried to be reconstituted before use.
